(19) United States
(12) Patent Application Publication
(10) Pub. No.: US 2023/0218663 A1
(43) Pub. Date: Jul. 13, 2023

(54) COMPOSITIONS AND METHODS FOR TREATING A SARS-COV-2 INFECTION

(71) Applicant: THE UNIVERSITY OF HONG KONG, Hong Kong (CN)

(72) Inventors: Hongzhe SUN, Hong Kong (CN); Kwok-Yung YUEN, Hong Kong (CN); Shuofeng YUAN, Hong Kong (CN); Runming WANG, Hong Kong (CN); Hongyan LI, Hong Kong (CN); Fuk-Woo CHAN, Hong Kong (CN); Tianfan CHENG, Hong Kong (CN); Suyu WANG, Hong Kong (CN)

(21) Appl. No.: 18/000,491

(22) PCT Filed: May 31, 2021
(86) PCT No.: PCT/CN2021/097317

§ 371 Date: Dec. 1, 2022

Related U.S. Application Data
(60) Provisional application No. 63/032,888, filed on Jun. 1, 2020.

(51) Int. Cl. A61K 33/245; A61P 31/14; A61K 45/06

(52) U.S. Cl. CPC A61K 33/245; A61P 31/14; A61K 45/06; A61K 9/0053

(57) ABSTRACT

Compositions and methods for treating a subject for a SARS-CoV-2 infection in a subject in need thereof are disclosed. The composition includes one or more Bismuth (III)-containing compounds, an analog thereof, or pharmaceutically acceptable salt thereof in a pharmaceutically acceptable carrier, and are used alone or in combination with a thiol-containing small molecule compound. Exemplary Bismuth (III)-containing compounds include Colloidal Bismuth Subcitrate (CBS); ranitidine bismuth citrate (RBC); Bi (TPP) (TPP: tetraphenylporphyrinate); and Bi (TPyP) (TPyP: tetra (4-pyridyl) porphyrin). The disclosed compounds and compositions can be used to treat a SARS-CoV-2 infection in a subject in need thereof. The compositions can be administered to a subject presently suffering from an infection of the SARS-CoV-2, who is exhibiting one or more symptoms of COVID-19. The compositions can also be administered to a subject that has been exposed to the SARS-CoV-2 but is asymptomatic.

### CROSS REFERENCE TO RELATED APPLICATIONS

This application claims the benefit of the filing dates of U.S. Provisional Application No. 63/032,888, filed on Jun. 1, 2020, the entire contents of which are incorporated by reference herein.

### FIELD OF THE INVENTION

This invention is generally in the field of compositions and methods for treating a SARS-CoV-2 infection.

### BACKGROUND OF THE INVENTION

The current pandemic of coronavirus disease 2019 (COVID-19) caused by the novel severe acute respiratory syndrome coronavirus 2 (SARS-CoV-2)¹ represents a global public health crisis, leading to around five million confirmed cases including 323,000 deaths globally. This emergency posts an unprecedented challenge to rapidly identify effective drugs for prevention and treatment. Similar to other coronaviruses2, SARS-CoV-2 synthesizes a battery of viral enzymes and proteins that are essential for viral entry, replication and pathogenesis, including structural proteins and nonstructural proteins (nsp). Intervention on viral entry or replication allows either vaccine or therapeutics to be developed3-6. Several enzymes7-9 including RNA-dependent RNA polymerase, 3-chymotrypsin-like protease as well as papain-like protease may serve as promising targets of potential therapeutic drugs.

Repurposing drugs already in clinical use for the treatment of COVID-19 is the only practical approach given the urgency and severity of the pandemic^{10,11}. Remdesivir, a broad-spectrum antiviral medication, has been reported to show efficacy against SARS-CoV-212. Remdesivir exhibited genuine but not dramatic benefits, with a median time to patient recovery reduced by about 4 days from 15 days, as well as a reduced mortality rate13. In severe COVID-19 patients, however, no significant clinical benefits of remdesivir treatment were observed14. Overall, current clinical trials on a series of antiviral agents indicate the big challenge to improve the clinical outcomes of COVID-19 patients^{10,15}. Therefore, it is of utmost urgent need for renewed efforts. There is still a need for compositions that can effectively treat COVID-19.

It is an object of the present invention to provide compositions for treating symptoms associated with a SARS-CoV-2 infection.

It is also an object of the present invention to provide methods for treating one or more symptoms associated with a SARS-CoV-2 infection.

### SUMMARY OF THE INVENTION

Compositions and methods for treating a subject for a SARS-CoV-2 infection in a subject in need thereof are disclosed. The composition include one or more Bismuth (III)-containing compounds, an analog thereof, or pharmaceutically acceptable salt thereof in a pharmaceutically acceptable carrier, alone, or in combination with a thiol-containing small molecule. A preferred thiol-containing small molecule is NAC. In one preferred embodiment, the compositions include one or more compounds selected from the group consisting of:

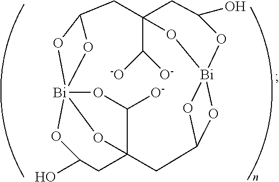

### Colloidal Bismuth Subcitrate;

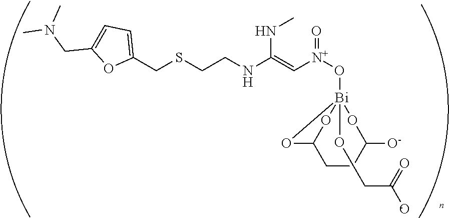

ranitidine bismuth citrate (RBC):

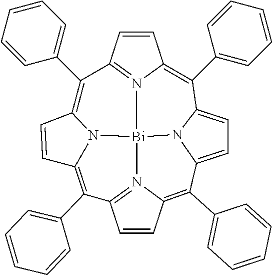

Bi(TTP) (TPP: tetraphenylporphyrinate); and

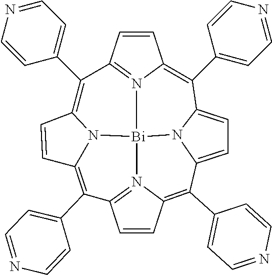

Bi(TPyP) (TPyP: tetra(4-pyridyl)porphyrin).

The Bismuth (III)-containing compound or pharmaceutically acceptable salt thereof is administered alone or preferably in combination with a thiol-containing small molecule, to reduce one or more symptoms of a disease, disorder, or illness associated with a SARS-CoV-2 infection. A preferred thiol-containing small molecule is NAC.

Also disclosed are method of treating a SARS-CoV-2 infection in a subject in need thereof. The methods include administering to the subject a composition comprising one or more Bismuth (III)-containing compounds, an analog thereof, or pharmaceutically acceptable salt thereof in a pharmaceutically acceptable carrier, alone, or in combination with a thiol-containing small molecule, in a therapeutically effective amount to reduce one or more symptoms of a SARS-CoV-2 infection. In a preferred embodiment, the treatment is effective to inhibit the helicase protein of SARS-CoV-2, in the subject, i.e., the compositions are administered in an effective amount to inhibit the helicase protein of SARS-CoV-2, in the subject.

The treatment is effective to reduce one or more symptoms associated with COVID 19, including, but not limited to fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhea, tiredness, nausea, vomiting, and combinations thereof.

The compositions can be administered to a presently suffering from an infection of the SARS-CoV-2, optionally, who is exhibiting one or more symptoms of COVID-19. The compositions can also be administered to a subject that has been exposed to the SARS-CoV-2, but is asymptomatic.

### BRIEF DESCRIPTION OF THE DRAWINGS

FIGS. 1A-1H show results of VeroE6 and Caco2 cells infected with SARS-CoV-2 (0.1 MOI) and treated with different concentrations of Pylorid and related compounds as indicated. Intracellular viral loads were detected at 48 hpi and normalized by human β-actin (FIGS. 1A-1D for Vero E6 cells and FIGS. 1E-1H for Caco2 cells). FIGS. 1M-1P. Virus copies in the cell culture supernatant were determined at 48 hpi by qRT-qPCR (FIGS. 1I-1L for Vero E6 cells and FIGS. 1M-1P for Caco2 cells).

FIG. 2A. Quantification of antigen-positive cells from randomly selected 800×800-pixel fields (n=4) over two independent experiments (one-way analysis of variance, AN A time-of-drug-addition assay, performed to determine the steps of the viral replication cycle targeted by each of the four drug compounds. FIG. 2B is a the scheme showing the experimental design, indicating the period of cell-compound incubation. Virus absorption was performed at ˜0-1 h (MOI=2), followed by replacement of fresh medium supplemented with the tested drug or DMSO. FIG. 2C show bar charts that show the virus yields in the supernatant of all groups, quantified by qRT—PCR at 9 h.p.i. One-way ANOVA was used to compare the treatment groups with the negative control group (0 μM, 0.1% DMSO). OVA). NP indicates the nucleocapsid protein of SARS-CoV-2. FIG. 2D. HEK293-hACE2 stable cells were infected with pseudo-SARS-CoV-2-Luc in the presence of DMSO or drug compounds, as indicated (n=3). Luciferase activity was measured at 48 h.p.i. and normalized as percent of DMSO control. One-way ANOVA was used to compare the treatment groups with the negative control group (0 μM, i.e. 0.1% DMSO). The results are shown as mean±standard deviations (S.D.). *p indicates <0.05, **p<0.01 and ***p<0.001. All the experiments were performed in triplicate and replicated twice. FIG. 2E. Virus copy in the cell culture supernatant after the treatment of ranitidine on SARS-CoV-2-infected VeroE6 cells (0.1MOI, 48 hpi). One-way ANOVA was used to compare the treatment groups with the negative control group (0 μM, i.e. 0.1% DMSO). The results are shown as mean±standard deviations (S.D.). n.s. indicates non-significant. FIG. 2F. Vero E6 and Calu-3 cells were infected with pseudo-SARS-CoV-2-Luc in the presence of vehicle (water) or RBC (500 μM). Vero E6 and Calu-3 cells were infected with pseudo-SARS-CoV-2-Luc in the presence of vehicle (water) or RBC (500 μM). Luciferase activity was measured at 48 h post-infection and normalized as % of the infected cells without any treatment. The results are shown as mean±S.D of n=3 biologically independent samples. Statistical significance was calculated using an unpaired two-tailed Student's t-test, **p<0.01 using Student's t-test. Grey dashed line represents mean value of the RBC-treated Vero E6 group, which is for easier comparison with that of the RBC-treated Calu-3 group.

FIG. 3A-3B show studies on Hamsters (n=5) were intranasally inoculated with 1000 PFU of SARS-CoV-2 and were intraperitoneally given either Pylorid, or Remdesivir or PBS for consecutive four days with the first dose given at 6 hpi. At 4 dpi, respiratory tissue viral yields in the nasal turbinate and lung tissues of the hamsters were detected by qRT-PCR assay (FIG. 3A) and TCID₅₀ assay (FIG. 3B), respectively. (FIG. 3C) Representative chemokine and cytokine of the lung tissues of the indicated groups as detected in the lung tissue homogenate at 4 dpi. The results are shown as mean value±SD. *p<0.05, **p<0.01, and ***p<0.001 when compared with the DMSO group. One-way ANOVA. FIG. 3D-3E show the purification of SARS-CoV-2 helicase. FIG. 3D The SDS-PAGE gel of SARS-CoV-2 helicase. The left lane: marker; the right lane: helicase FIG. 3E Size-exclusion chromatography profile of SARS-CoV-2 helicase. FIG. 3F. Histological score indicating lung histopathological severity in each group. The scoring method is detailed in the Methods. Data are presented as mean±s.d. of four randomly selected slides from each group. Unpaired two-tailed Student's t-test, ***P<0.001 when compared with the DMSO control group. The histological score of mock infection was set as zero.

FIGS. 4A-4D show inhibition of ATPase activity of the SARS-CoV-2 helicase by Pylorid and related compounds at varying concentrations as indicated. FIGS. 4E-4H. Titration of the DNA-unwinding activity of the SARS-CoV-2 helicase by Pylorid and related compounds at varying concentrations as indicated by a FRET-based assay. FIGS. 4A-4H. The data are expressed as a percentage of the control reaction in the absence of inhibitors. Dose-response curves for half-maximum inhibitory concentration (IC₅₀) values were determined by nonlinear regression. FIGS. 4I-4H. Restoration of activity of (i) ATPase and (FIG. 4J) DNA-unwinding activity of Bi-SARS-CoV-2 helicase upon supplementation of various ratios of zinc(II) as indicated. (FIGS. 4A-4J) All the assays were performed in triplicate and the data are shown as mean±SD. (FIGS. 4K-4L) Lineweaver-Burk plots showing the kinetics inhibition of (FIG. 4K) ATPase activity and (FIG. 4L) DNA-unwinding activity of SARS-CoV-2 helicase by Pylorid. The effect of the inhibitors on the enzyme was determined from the double reciprocal plot of 1/rate (1/V₀) vs. 1/substrate concentration in the presence of varying concentrations of Pylorid. The Kᵢ values were calculated by the intersection of the curves obtained by plotting 1/V vs. the inhibitor concentration.

FIG. 5A shows different UV-vis spectra of titration of different molar equivalents of bismuth(III) to apo-SARS-CoV-2 helicase. The inset shows a titration curves plotted at ˜340 nm against the molar ratio of [bismuth(III)]/[apo-SARS-CoV-2 helicase]. The assays were performed twice and the representative data were shown. (FIG. 5B) The substitution of zinc(II) in SARS-CoV-2 helicase by Pylorid over equilibrium dialysis. The metal contents of zinc(II) and bismuth(III) were determined by ICP-MS. Mean value of three replicates are shown and error bars indicate±SD.

FIG. 6A-6I. NAC stabilizes and promotes absorption of bismuth drug CBS in vitro and in vivo. FIG. 6A shows in vitro chemical stability of CBS (2.5 mM) at pH 1.2 (left), pH 7.4 (middle) and pH 9.2 (right) in the presence of escalating amounts of NAC. The percentage of remaining bismuth was calculated from the ratio of bismuth content in supernatant measured at 1 h to 0 h (n=3). (FIG. 6B) Cumulative amount of bismuth in acceptor compartments at acidic iso-pH 1.2 for three bismuth drugs in the absence and presence of appropriate amounts of NAC using a PAMPA permeability assay (n=3). (FIG. 6C) Cumulative amount of bismuth in acceptor compartments over time for CBS (150 μM) in the absence and presence of 10NAC (1.5 mM) in a Caco-2 cell monolayer model (n=3). (FIG. 6D) Bismuth accumulation in Caco-2 cell monolayer (n=3). (FIG. 6E) The apparent permeability coefficient (Pₐₚₚ, cm/s) of CBS (150 μM) and CBS (150 μM)+10NAC (1.5 mM) through the Caco-2 monolayer (n=3). (FIG. 6F) Cumulative amount of bismuth transported through duodenum verse time for CBS (200 μM) in the presence of escalating amounts of NAC in the everted rat intestinal sac model (n=3). (FIG. 6G) Blood bismuth concentrations at 1-hour and 2-hour after oral administration to Balb/c mice of CBS (150 mg/kg) in the presence of escalating amounts of NAC (n=3). (FIG. 6H) Mean blood bismuth concentration versus time profile of CBS and CBS (150 mg/kg)+10NAC (610 mg/kg) after oral administration in SD rats (n=5 for each time interval). (FIG. 6I) Distribution of bismuth in different organs after oral administration of CBS and CBS+10NAC in SD rats (n=5). The samples were collected at 24-hour after drug administration from the same batch of rats in (FIG. 6H). (FIG. 6A-I) Measurement of drug concentration were based on metal content by using ICP-MS. Data are shown as mean±SD. Statistical significance was calculated using an unpaired two-tailed Student's t-test, ***P<0.001, **P<0.01, *P<0.05.

FIG. 7A-7D show the effect CBS+3NAC on replication of human-pathogenic coronaviruses in human cellular models in a dose-dependent manner (n=3). FIG. 7A. SARS-CoV-2 in Vero E6 cells. FIG. 7B. SARS-CoV-2 (B.1.1.7 variant) in Vero E6 cells. FIG. 7C. MERS-CoV in Vero E6 cells. (IG. 7D. HCoV-229E in HELF cell. Viral load in the cell culture supernatant was quantified by qPCR with reverse transcription (RT-qPCR). Data are shown as mean±SD. All statistical analyses were compared with the control group (0 μM) and significance was calculated using an unpaired two-tailed Student's t-test, ***P<0.001, **P<0.01, *P<0.05. FIG. 7E. Quantification of NP -positive cells from randomly selected 800×800-pixel fields (n=4) over two independent experiments (one-way analysis of variance, ANOVA). ****P<0.0001, **P<0.01. Data are shown as mean±SD. FIG. 7F. Virus yields in the supernatant of all groups in a time-of-drug-addition assay, quantified by qRT-PCR at 9 h.p.i. (n=3). Data are shown as mean±SD. One-way ANOVA was used to compare the treatment groups with the vehicle control group (0 μM). ****P<0.0001, **P<0.01, *P<0.05. FIG. 7G. Scheme depicting the therapeutic treatment via oral administration of vehicle, CBS (300 mg/kg), NAC (370 mg/kg) and CBS (300 mg/kg)+3NAC (370 mg/kg), given at Day −2, −1, 0 and 1 and the hamsters were challenged by virus at Day 0; Tissue samples were collected at two days after infection. FIG. 7H. Viral yield in hamster lung tissue (n=8). FIG. 7I. Cytokine IL-6 gene expression level. FIG. 7H-7I. Data are shown as mean±SD. Statistical significance was calculated using Kruskal-Wallis with Dunn's multiple comparison test. ***P<0.001. FIG. 7J) Quantification of NP-positive cells from randomly selected 800×800-pixel fields (n=4) in lung tissue (one-way analysis of variance, ANOVA). ****P<0.0001, *P<0.05. Data are shown as mean±SD. FIG. 7K) Semiquantitative histology scores were given to each lung tissue by grading the severity of damage in bronchioles, alveoli and blood vessels and accumulating the total scores. The histological score of mock infection was set as zero. Data are shown as mean±SD. Statistical significance was calculated using an unpaired two-tailed Student's t-test, ****p<0.0001, **P<0.01, *P<0.05. FIG. 7L Virus copies in the Vero E6 cell culture supernatant after NAC treatment (n=3). Data are shown as mean±SD. No difference in statistical significance was found among groups using an unpaired two-tailed Student's t-test. FIG. 7M Bismuth accumulation in lung after oral administration of CBS (150 mg/kg)+10NAC (610 mg/kg) in Balb/c mice (n=3) for 1 day, consecutive 2 days, consecutive 3 days. Data are shown as mean±SD. No difference in statistical significance was found among groups using an unpaired two-tailed Student's t-test.

FIG. 8A-8L. Bismuth drug exhibits antiviral potency through targeting multiple conserved key cysteine proteases/enzymes in SARS-CoV-2. (A-D) Inhibition of CBS+3NAC on (FIG. 8A) dsDNA unwinding activity of SARS-CoV-2 Hel (FIG. 8B) ATPase activity of the SARS-CoV-2 Hel (FIG. 8C) SARS-CoV-2 PL^{pro} activity (FIG. 8D) SARS-CoV-2 M^{pro} activity (n=3). (FIG. 8E-F) Lineweaver-Burk plots showing the kinetics of CBS+3NAC inhibition on (FIG. 8E) SARS-CoV-2 PL^{pro} activity (FIG. 8F) SARS-CoV-2 M^{pro} activity. The effect of CBS+3NAC on the enzymes was determined from the double reciprocal plot of 1/rate (1/V) versus 1/substrate concentration in the presence of varying concentrations of CBS+3NAC. The Ki values were calculated by the intersection of the curves obtained by plotting 1/V versus inhibitor concentration for each substrate concentration. (FIG. 8G-8H) Dependence of absorbance at 340 nm verse time for the reaction of Bi³⁺ (20 mol eq.) with (G) SARS-CoV-2 PL^{pro} and (FIG. 8H) SARS-CoV-2 M^{pro}. The curve is shown as a nonlinear least square fit using an one-phase exponential function. (FIG. 8I-8J) Difference UV-vis spectra for titration of various molar equivalents of Bi³⁺ with (FIG. 8I) apo-SARS-CoV-2 PL^{pro} and (FIG. 8J) SARS-CoV-2 M^{pro}. The insets shows a titration curve plotted at ˜340 nm against the molar ratio of (FIG. 8I) [Bi³⁺]/[apo-SARS-CoV-2 PL^{pro}] and (J) [Bi³⁺]/[SARS-CoV-2 M^{pro}]. The assays were performed twice and representative data are shown. (FIG. 8K) Released Zn²⁺ from SARS-CoV-2 PL^{pro} after incubation with Bi³⁺ at escalating concentrations (n=3). (FIG. 8L) Semi-quantification of free cysteine in SARS-CoV-2 M^{pro} after incubation with Bi³⁺ on an Ellman's assay (n=3). (FIG. 8A-8D, 8K, 8L) Data are shown as mean±SD.

FIG. 9A-9C. Oral administration of CBS+3NAC exhibits reversible pathological change in mice kidney, as revealed by (FIG. 9A) Body weight changes verse time (FIG. 9B) BUN level versus time (n=4) (FIG. 9C) creatinine level verse time (n=4). Data are shown as mean±SD. No difference in statistical significance was found among groups using an unpaired two-tailed Student's t-test.

FIG. 10A shows inhibition of CBS on SARS-CoV-2 PL^{pro} activity. FIG. 10B SARS-CoV-2 M^{pro} activity (n=3). Data are shown as mean±SD.

FIG. 11 shows the effect on SARS-CoV-2 in Vero E6 cells following coadministration of bismuth drugs and thiol containing drugs. Data are shown as mean±SD. All statistical analyses were compared with the control group (0 μM) and significance was calculated using an unpaired two-tailed Student's t-test, **P<0.01.

### DETAILED DESCRIPTION OF THE INVENTION

### I. Definitions

“Carrier” or “excipient”, as used herein, refers to an organic or inorganic ingredient, natural or synthetic inactive ingredient in a formulation, with which one or more active ingredients are combined.

“Therapeutically effective” or “effective amount” as used herein means that the amount of the composition used is of sufficient quantity to ameliorate one or more causes or symptoms of a disease or disorder. Such amelioration only requires a reduction or alteration, not necessarily elimination. As used herein, the terms “therapeutically effective amount” “therapeutic amount” and “pharmaceutically effective amount” are synonymous. One of skill in the art can readily determine the proper therapeutic amount.

“Individual”, “host”, “subject”, and “patient” are used interchangeably herein, and refer animals, particularly mammals, including, but not limited to, primates such as humans.

“Pharmaceutically acceptable”, as used herein, means a non-toxic material that does not interfere with the effectiveness of the biological activity of the active ingredients.

“Pharmaceutically acceptable salt”, as used herein, refers to derivatives of the compounds defined herein, wherein the parent compound is modified by making acid or base salts thereof.

“Treatment”, as used herein, refers to the medical management of a patient with the intent to cure, ameliorate, stabilize, or prevent a disease, pathological condition, or disorder.

### II. Compositions

Bismuth (III) containing compounds are disclosed, the pharmaceutical formulations of which can be used to treat subjects infected with SARS-CoV-2, alone, but preferably, in combination with one or more thiol-containing small molecules.

A. Compounds

The disclosed compounds include containing compounds and thiol-containing small molecules.

(i) Bismuth (III) Containing Compounds

Preferred bismuth containing compounds for use in the disclosed methods include bismuth (III) citrate based drugs (complexes of Bismuth (III) with citrate) or Bismuth (III) porphyrins. Specific examples include, but are not limited to Bismuth Subcitrate, specifically, colloidal bismuth subcitrate (CBS)

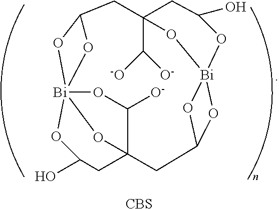

CBS is a complex bismuth salt of citric acid which is soluble in water but precipitates at pH less than 5. in gastric juice the optimum pH for precipitation is 3.5. CBS can be formulated into pharmaceutical compositions in solid dosage form for oral administration, such as tablets and capsules.

or

ranitidine bismuth citrate (RBC:

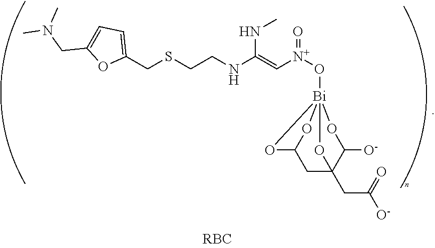

Bi(TPP) (TPP: tetraphenylporphyrinate)

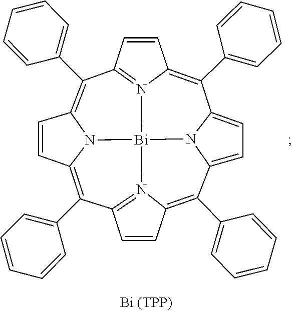

and
Bi(TPyP) (TPyP: tetra(4-pyridy)porphyrin)

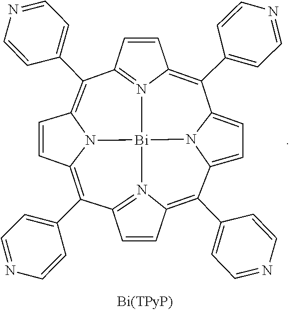

(ii) Thiol-Containing Small Molecules

Thiol-containing small molecules preferably have molecular weight of less than 2,000 Daltons, more preferably less than 1,500 Daltons, most preferably less than 1,000 Daltons.

A preferred thiol-containing small molecule is N-acetyl cysteine (NAC). NAC is an FDA-approved drug that is commonly used as a mucolytic in patients with pneumonia, as well as various other medical conditions such as paracetamol overdose⁽¹²⁾. NAC is available as an intravenous (IV), oral, and inhaled drug^{(13, 14)}. It is generally safe with few side effects clinically, and also exhibits anti-oxidant, anti-inflammatory, and immunomodulating effects^{(15, 16)}. However, other thiol-containing compounds that include thio, thiol, aminothiol or thioester moiety are known. Examples include, glutathione (GSH), penicillamine (PCM), captopril (CPL), and thiosalicylic acid (TSA), sodium thiosulfate (STS), GSH ethyl ester, D-methionine, dimecarprol, D-β,β-dimethylcysteine and thiol amifostine (Ethyol or WR2721).

B. Formulations

The disclosed Bismuth (III) compounds or pharmaceutically acceptable salts thereof can be formulated in a pharmaceutical formulation. The thiol-containing small molecules can be formulated in the same pharmaceutical formulation as the Bismuth (III) compounds or pharmaceutically acceptable salts thereof, and in this embodiment, will include an effective amount of thiol-containing small molecule to stabilize the Bismuth (III) compounds or pharmaceutically acceptable salts thereof at low pH, for example, pH 1.2. The thiol-containing small molecules can be formulated in a separate pharmaceutical formulation.

Pharmaceutical formulations can be for administration by parenteral (intramuscular, intraperitoneal, intravenous (IV) or subcutaneous injection), enteral administration.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, desintegrators, fillers, and coating compositions.

In one preferred embodiment, the formulation is in the form or a tablet or capsule or a colloidal suspension. In another preferred embodiment, the compound is in a form suitable for intramuscular or intravenous injection. A swallowoable (tablet) form of CBS is disclosed in WO 1999011848. Ranitidine bismuth citrate may conveniently be formulated as tablets (including chewable tablets), capsules (of either the hard or soft type), or as a liquid preparation, as described for example in UK Patent. Nos. 2220937A and 2248185A. Tablets are generally preferred. Thus, the composition may be prepared by conventional means with additional carriers or excipients such as binding agents (e.g., pregelatinised maize starch, polyvinylpyrrolidone or hydroxypropylmethylcelose); fillers (e.g. lactose, microcrystalline cellulose or calcium hydrogen phosphate); lubricants (e.g. magnesium stearate, talc or silica); disintegrates (e.g. starch or sodium starch glycollate); or wetting agents (e.g. sodium lauryl sulphate). An alkaline salt of the type described in UK Patent Specification No. 2248185A may be added to improve the rate of disintegration and/or dissolution of the composition. Tablets may be coated by methods well known in the art. The preparations may also contain flavouring, colouring and/or sweetening agents as appropriate.

Tablets may be prepared. for example, by direct compression of such a mixture, Capsules may be prepared by filling the blend along with suitable excipients into gelatin capsules, using a suitable filling machine.

1. Parenteral Formulations

The compounds described herein can be formulated for parenteral administration.

For example, parenteral administration may include administration to a patient intravenously, intradermally, intraarterially, intraperitoneally, intralesionally, intracranially, intraarticularly, intraprostatically, intrapleurally, intratracheally, intravitreally, intramuscularly, subcutaneously, subconjunctivally, intravesicularly, intrapericardially, intraumbilically, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, using a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

(a) Controlled Release Formulations

The parenteral formulations described herein can be formulated for controlled release including immediate release, delayed release, extended release, pulsatile release, and combinations thereof.

i. Nano- and Microparticles

For parenteral administration, the one or more compounds, and optional one or more additional active agents, can be incorporated into microparticles, nanoparticles, or combinations thereof that provide controlled release of the compounds and/or one or more additional active agents. In embodiments wherein the formulations contain two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulo se, and carboxymethyl-cellulose), alginic acid, lactose and talc. Additionally, a pharmaceutically acceptable surfactant (for example, lecithin) may be added to facilitate the degradation of such microparticles. Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

ii. Method of Making Nano- and Microparticles

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art. p For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

2. Enteral Formulations

Suitable oral dosage forms include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art. Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

(a) Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation into the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

(i) Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulo se, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename EUDRAGIT t®. In further preferred embodiments, the acrylic polymer comprises a mixture of two acrylic resin lacquers commercially available from Rohm Pharma under the tradenames EUDRAGIT® RL30D and EUDRAGIT RS30D, respectively. EUDRAGIT® RL30D and EUDRAGIT RS30D are copolymers of acrylic and methacrylic esters with a low content of quaternary ammonium groups, the molar ratio of ammonium groups to the remaining neutral (meth)acrylic esters being 1:20 in EUDRAGIT RL30D and 1:40 in EUDRAGIT® RS30D. The mean molecular weight is about 150,000. EUDRAGIT S-100 and EUDRAGIT ® L-100 are also preferred. The code designations RL (high permeability) and RS (low permeability) refer to the permeability properties of these agents. EUDRAGIT RL/RS mixtures are insoluble in water and in digestive fluids. However, multiparticulate systems formed to include the same are swellable and permeable in aqueous solutions and digestive fluids.

The polymers described above such as EUDRAGIT RL/RS may be mixed in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

(ii). Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used Small quantities of an anti-foaming agent, such as a silicone (e.g., simethicone), may also be added to the coating composition.

As will be appreciated by those skilled in the art and as described in the pertinent texts and literature, several methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of several conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### III. Methods of Use

Methods of treating a SARS-CoV-2 infection in subject in need thereof are provided. The subject can be, a mammal for example, a human. The subject is preferably, a human subject. The compositions are administered to reduce one or more symptoms associated with a SARS-CoV-2 infection. In a preferred embodiment, the composition is administered in an effective amount to inhibit the SARS-CoV-2 helicase.

The administered compositions in one embodiment is a formulation of one or more bismuth (III) compounds or pharmaceutically acceptable salts thereof as disclosed herein, a formulation of one or more bismuth (III) compounds or pharmaceutically acceptable salts and one or more thiol-containing small molecules wherein the and one or more thiol-containing small molecules are in an effective amount to stabilize the Bismuth (III) compounds or pharmaceutically acceptable salts thereof at low pH, for example, pH 1.2. In other embodiments.

In other embodiments, the treatment includes administering to the subject a formulation one or more bismuth (III) compounds or pharmaceutically acceptable salts thereof as disclosed herein and a formulation of a thiol-containing small molecules, administered concurrently, or sequentially. In this embodiment, the thiol-containing small molecules administered is effective to stabilize the Bismuth (III) compounds or pharmaceutically acceptable salts thereof at low pH, for example, pH 1.2. A thiol-containing small molecule administered is effective to stabilize the Bismuth (III) compounds or pharmaceutically acceptable salts thereof at low pH, for example, pH 1.2 is used at a 3 or 10 mol eq. to the bismuth (III) compounds or pharmaceutically acceptable salts thereof.

A preferred thiol-containing small molecule is NAC.

The subject has, in some embodiments been, or will be, exposed to the virus. In preferred embodiments, the subject has been exposed to the virus or is experiencing an active viral infection, identified by one or more symptoms associated with COVID 19. Symptoms include, but are not limited to, fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhea, tiredness, nausea, vomiting, and combinations thereof, and the subject may or may not be diagnosed as having a SARS-CoV-2 infection, using known methods for testing subject for infection with SARS-CoV-2. The current gold standard for molecular diagnosis of COVID-19 is based on the detection of SARS-CoV-2 RNA by real-time quantitative reverse transcription-polymerase chain reaction (qRT-PCR).

The compositions can also be administered prophylactically to, for example, reduce or prevent the effects of future exposure to virus and the infection that may associated therewith. Thus, in some embodiments, the subject has not been exposed to the virus and/or is not yet experiencing an active viral infection. In some embodiments, the subject is a healthy subject. In a particularly preferred embodiment, however, the composition is administered to ameliorate one or more symptoms of COVID 19, in the subject.

The disclosed methods will be understood by the following non-limiting examples.

In one embodiment the subject is administered a composition containing a bismuth (III) compound, in an effective amount to reduce one or more symptoms of COVID 19. In a preferred embodiment, the composition contains a Bi(III) citrate based drug (complexes of Bismuth (III) with citrate) or Bismuth (III) porphyrins.

For example, the subject can be administered a composition containing an effective amount of a bismuth compound alone or in combination with a thiol-containing small molecule such as NAC, including but not limited to Bismuth Subcitrate, for example, colloidal bismuth subcitrate (CBS)

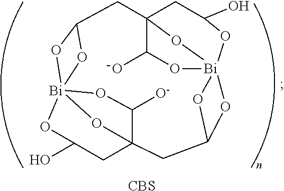

or

ranitidine bismuth citrate (RBC:

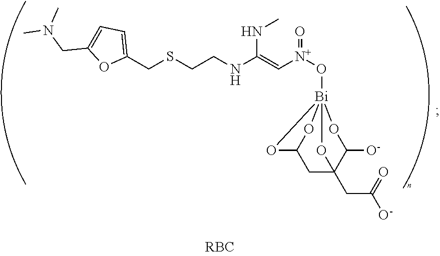

In other embodiments, the subject can be administered a composition containing a Bismuth (III) porphyrin compound, for example, Bi(TPP) (TPP: tetraphenylporphyrinate)

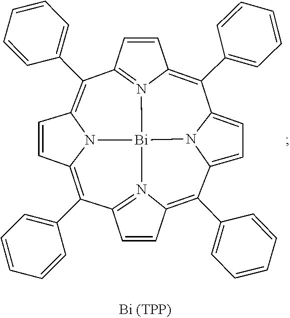

or
Bi(TPyP) (TPyP: tetra(4-pyridyl)porphyrin)

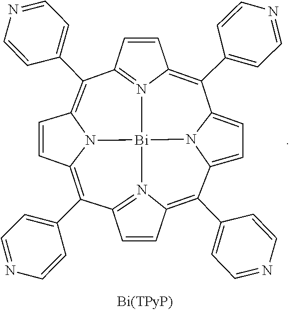

The subject can be treated with one or more of the disclosed compounds, the treatment regime being effective reduce one or more symptoms of COVID 19, preferably, the treatment regime being effective to inhibit SARS-CoV-2 helicase.

The thiol-containing small molecules are administered an amount effective to increase blood concentration of bismuth, prolong Tmax and increase AUC_{0→12 h} in the subject, when compared to administration of the bismuth containing compounds in a control subject, without one or more thiol-containing small molecules.

The disclosed methods will be understood by the following non-limiting examples.

### EXAMPLES

### Materials and Methods

Chemicals, Virus and Cell Lines

Colloidal bismuth citrate (De-Nol/CBS) and ranitidine bismuth citrate (Pylorid) were kindly provided by Livzon Pharmaceutical Group. The RBC used in the current study possessed the composition ranitidine:Bi(iii):citrate=1:1:1 with the molecular formula C₁₉H₂₇N₄O₁₀SBi²³. Bi(TPP), Bi(TPyP) and Bi(NTA) were prepared as previously described³⁶. Auranofin was purchased from MedChemExpress (MCE). The combination of CBS and NAC was freshly prepared before experiments by physically mixing CBS and appropriate molar equivalents of NAC, followed by adjustment of pH in the range of 5-6 with 0.05 M NaOH. Bismuth subsalicylate (BSS) and bismuth subgallate (BGS) were obtained from Alfa Aesar. Bi(NTA)₃ was prepared as previously described⁽¹⁾. Kanamycin sulfate and Luria-Bertani (LB) broth powder were purchased from Affymetrix. All the other chemicals were purchased from Sigma-Aldrich unless otherwise stated and used directly without further purification. Cell lines used in this study were chosen according to their sensitivity to replication of corresponding coronavirus.

The SARS-CoV-2 (HKU-001a strain) (GenBank accession number: MT230904) and B.1.1.7 variant (GISAID accession no.: EPI_ISL_1273444) were isolated from the nasopharyngeal aspirate specimen of a laboratory-confirmed COVID-19 patient in Hong Kong³⁷.

Human colon epithelial (Caco2) cells and African green monkey kidney (VeroE6) cells and human lung epithelial cells (Calu-3) were purchased from ATCC (without further authentication and confirmed to be free of mycoplasma contamination by PlasmoTest (InvivoGen)) and maintained in Dulbecco's Modified Eagle's Medium (DMEM) medium supplemented with 10% of Fetal Bovine Serum (FBS, Gibco, Paisley, UK), 1% of penicillin-streptomycin (Gibco BRL, Grand Island, N.Y., USA) and 1% of non-essential amino acids (Gibco BRL, Gibco, Grand Island, N.Y., USA).

Monkey Vero E6 cells (ATCC, CRL-1586) were cultured in DMEM supplemented with 10% FBS, 50 U mL⁻¹ penicillin and 50 μg mL⁻¹ streptomycin. Human embryonic lung fibroblasts (HELF) were developed in-house. Cells were maintained at 37° C., in an atmosphere of 5% CO₂ and 90% relative humidity.

SARS-CoV-2 pseudoviral particles (replication-deficient murine leukemia virus (MLV) pseudotyped with the SARS-CoV-2 spike protein) were purchased from eEnzyme (cat no. SCV2-PsV-001). The MERS-CoV strain (HCoV-EMC/2012) was a gift from Dr. Ron Fouchier (3). The hCoV-229E strain is from a previous collection^{(4,5)}. All experiments with live viruses were conducted using biosafety level 3 facilities in Queen Mary Hospital, The University of Hong Kong, as previously described³⁸.

Cell Viability Assay

The CellTiterGlo® luminescent assay (Promega Corporation, Madison, Wis., USA) was performed to detect the cytotoxicity of the selected compounds as previously described³⁹. Briefly, VeroE6 cells (4×10⁴ cells per well) and Caco2 cells (4×10⁴ cells per well) were incubated with different concentrations of the individual compound in 96-well plates for 48 hrs, followed by the addition of the substrate and measurement of luminance 10 min later. Ridaura and Au(PEt3)Cl were dissolved in DMSO, and the final percentage of DMSO was kept at 1% in culture medium. The 50% cytotoxicity concentration (CC₅₀) values of the drug compounds were calculated by Sigma plot (SPSS) in an Excel add-in ED50V10.

Animals

All experiments were approved by, and performed in accordance with the guidelines approved by Committee on the Use of Live Animals in Teaching and Research (CULATR) of the University of Hong Kong.

For mouse experiments, 6-to-8-week-old female BALB/c mice with body weight of 18-22 g, were purchased from Charles River Laboratories, Inc. All animal procedures were approved by CULATR of the University of Hong Kong (reference code: CULATR 5079-19). For rat related experiments, Sprague Dawley rats with body weight of 200-220 g, were supplied by the Laboratory Animal Services Center at the Chinese University of Hong Kong. The animal experiments were conducted under the approval of Animal Ethics Committee of the Chinese University of Hong Kong (reference code: 19/074/GRF-5-C & (19-589) in DH/SHS/8/2/1 Pt.22) All the animals were randomly caged in biosafety level housing and given access to standard pellet feed and water ad libitum before the commence of corresponding experiments.

For hamster experiments, 6—to-10-week-old male and female Syrian hamsters with body weight of 70-100 g, were obtained from the Chinese University of Hong Kong Laboratory Animal Service Centre through the HKU Centre for Comparative Medicine Research. The hamsters were kept in biosafety level 2 housing and given access to standard pellet feed and water ad libitum, as previously described (2, 7). All experimental protocols were approved by the CULATR of the University of Hong Kong and were performed according to the standard operating procedures of the bio safety level 3 animal facilities (reference code: CULATR 5370-20).

### Analyses of Bismuth by Inductively Coupled Plasma Mass Spectrometer (ICP-MS)

ICP-MS was used to monitor the levels of bismuth in all investigated subjects. A quadrupole-based inductively coupled plasma mass spectrometer (ICPMS) (Agilent 7700×, Agilent Technologies, Calif.), equipped with a glass concentric nebulizer was used in this study. The samples were diluted to an appropriate concentration, sprayed into aerosols using microconcentric nebulizer and introduced into the ICP directly for time-resolved ICP-MS measurements. Samples were further diluted when the measured signals exceeded the liner range of standard curve. Bismuth contents (Bi²⁰⁹) in the investigated substance were calculated according to the standard curve in 1% nitric acid or respective blank control solution of organ and blood. Only one isotope was monitored in each measurement.

The main parameters were listed as follows: RF power (1300 kW); spray chamber (Scott spray chamber); nebulizer (MicroMist nebulizer); lens: (Ni); nebulizer gas flow (0.8 mL min⁻¹); acquisition mode: TRA Time Resolved Analysis); dwell time: 100 ms; reaction gas (no gas); temperature (2° C.). Bismuth standard solutions were prepared by diluting Multielement Calibration Standard (Fluka Analytical, 90243). The internal standard (10 mg L⁻¹; Agilent Technologies, 5188-6525) was used during the measurement.

Chemical Stability

Simulated gastric fluid, phosphate buffered saline and sodium bicarbonate buffer were applied to mimic environment in pH 1.2, 7.4 and 9.2. The simulated gastric fluid was prepared by dissolving NaCl (0.2 g) and pepsin (0.32 g) in about 70 mL of deionized water. The pH was then adjusted to 1.2 with 10 M HCl. The volume was finally adjusted to 100 mL with deionized water. Phosphate buffered saline was made from 2.7 mM potassium chloride, 1.8 mM monopotassium phosphate, 137 mM sodium chloride and 10 mM disodium phosphate. The pH was adjusted to 7.4 with HCl. 150 mM Sodium bicarbonate solution was prepared by dissolving NaHCO₃ into deionized water and the pH was adjusted to 9.2.

To monitor the stability of bismuth-NAC in different pH, bismuth-NAC mixtures were prepared in ratios of 1:1, 1:3 and 1:10 by adding appropriate amount of NAC into 10 mM CBS solution. Each bismuth-NAC (500 μL) was mixed with the pH buffer (500 μL) and incubated for 24 hours. The mixtures were centrifuged, and each supernatant was aliquoted into separated tube as samples which were subsequently subjected to ICP-MS for the measurement of remaining bismuth content in the supernatant.

Bismuth-thiol mixtures were prepared by titration of NAC to solutions of bismuth drugs. Mixtures of Bi-NAC in a ratio of 1:3 were prepared by adding appropriate amounts of NAC into solution of CBS (10 mM) and RBC (10 mM), respectively. Mixtures of BSG and BSS with NAC were obtained by dissolving appropriate amounts of BSG and BSS powders in NAC (100 mM) with a molar ratio of 1:10, respectively. Mixtures of Bi-thiol in a ratio of 1:3 were prepared by adding appropriate amounts of reduced glutathione (GSH) and penicillamine (PCM) into CBS (10 mM) solution, respectively. A mixture of CBS and captopril (CPL) was made in a ratio of 1:10. For thiosalicylic acid (TSA), it was first dissolved with 10% DMSO and 30% PEG in deionized water, mixed with CBS solution in a ratio of 1:1. The bismuth-thiol mixtures (2 mL) were then added to the aforementioned pH buffer (2 mL). Photos were taken as a record and shown in FIG. S1.

Parallel Artificial Membrane Permeability Assay (PAMPA)

PAMPA was used to determine bismuth permeation in the absence or presence of NAC. Donor (apical) solutions were prepared by adding CBS (2.5 mM), CBS (2.5 mM)+1NAC (2.5 mM), CBS (2.5 mM)+3NAC (7.5 mM), CBS(2.5 mM)+10NAC (25 mM), CBS(2.5 mM)+20NAC (50 mM) in PBS (pH 1.2). About 5 μL of egg lecithin in dodecane (1% w/v) were added onto the artificial membrane of each well in the donor plate for the activation of the membrane. Subsequently, 400 μL of acceptor solution were added in each well of the acceptor plate (BioAssay System, US), and covered by the donor plate with an aliquot of 200 μL of donor solution in each well. The system was incubated 16-hour at room temperature, followed by the measurement of bismuth concentrations of each investigated substance in starting solution, donor solution after incubation by ICP-MS. The assay was performed in triplicate.

In vitro Caco-2 Permeability Assay

The in vitro permeability of CBS in the absence or presence of NAC was evaluated by using the Caco-2 permeability assay according to a method as described previously (8).

Briefly, Caco-2 cells with 80-90% confluence was sub-cultured by trypsinization with 0.05% trypsin-EDTA (Gibco BRL, Gibco, Grand Island, N.Y., USA) and plated onto six-well plates Transwell inserts (24 mm i.d., 0.4 μm pore size, 4.67 cm², polycarbonate filter, Corning Costar Co. NY, USA) coated with collagen (collagen type I rat tail solution, ST. Louis, Mo., USA) at a density of 1-2×10⁵ cells per well and cultured for 21 days prior to transport experiments. Transepithelial Electrical Resistance (TEER) value of each well was monitored by epithelial voltammeter (EVOM2, World Precision Instruments Inc., Berlin, Germany) with STX2 electrode set according to the manufacturer's instructions to ensure the integrity of the monolayer. Cell monolayer with TEER above 600 Ω cm² was used in this study.

For transport study, CBS (150 μM) and CBS (150 μM)+10NAC (1.5 mM) were prepared in transport buffer [Hank's balanced salt solution (pH 7.4, HBSS, Grand Island, N.Y., USA) with phenol red] and loaded in the donor (apical) chamber in a 1.5 mL aliquot, respectively, followed by adding 2.5 mL transport buffer in receiver (basolateral) chamber. Aliquots of 0.1 mL samples was withdrawn from the receiver chamber at different time intervals (10, 20, 30, 40, 50, 60 mM) and equal volume of blank transport buffer was supplemented in receiver chamber immediately. The assay was performed in triplicate. Samples collected from the transport study were diluted to appropriate concentrations with 1% HNO₃ followed by ICP-MS measurement of bismuth content transported from donor side to receiver side. At the end of transport study, Caco-2 cells on the monolayer were also collected after washing with PBS for six times, and the numbers of cells were counted by hemocytometer under an optical microscope. The resulting cell pellets were acidified with 69% HNO₃, and diluted appropriately for the measurement of bismuth accumulation in cells. The apparent permeability coefficients (Pₐₚₚ, cm/s) of CBS from different treatment groups were calculated through the following equation (9):

$$Papp = \frac{dQ}{dt} \times \frac{1}{A \times C}$$

where dQ/dt (μmol/s) is cumulative concentration at time t, C (μM) is the initial concentration of test drugs in the donor chamber and A (cm²) is the surface area of the monolayer.

Ex Vivo Everted Gut Sac Model

Ex vivo everted gut sac model was performed according to a modified method (10, 11). For the preparation of everted gut sac, small intestines were rapidly isolated from rats right after their sacrificing followed by being washed several times with 0.9% saline. Duodenum was segmented of the intestine (˜4 cm) in oxygenated medium [Krebs-Henseleit solution (pH 7.4, 1.25 mM NaHCO₃, pH 7.4, 5.9 mM NaCl, 23.5 μM KCl, 60 μM MgSO₄, 62.5 μM CaCl₂, 60 μM KH₂PO₄, 550 μM glucose)], gently everted, washed, slid onto a glass rod and fastened with braided silk. Duodenum was clamped at one end and filled with an aliquot of 1 mL fresh oxygenated medium, and subsequently sealed with a second clamp, resulting an everted gut sac with approximately 3 cm in length using braided silk sutures.

The everted gut sacs (n=3 per group) were dialyzed in oxygenated medium supplemented with CBS (200 μM), CBS (200 μM)+1NAC (200 μM), CBS (200 μM)+3NAC (600 μM), CBS (200 μM)+10NAC (2 mM), respectively, at 37° C. Aliquots of 50 μL samples were withdrawn from the gut sacs at different time intervals (15, 30, 45, 60 min) and equal volume of oxygenated medium was supplemented in gut sacs immediately. The length and width of each intestinal segment was measured after the final sample was taken. Bismuth content transported into the gut sacs was measured by ICP-MS as mentioned above.

In Vivo Pharmacokinetics Studies

To estimation the impact of NAC on blood bismuth content, groups of Balb/c mice (n=3 per group) were orally administered with CBS (150 mg/kg), CBS (150 mg/kg)+3NAC (180 mg/kg), CBS (150 mg/kg)+10NAC (610 mg/kg), CBS (150 mg/kg)+20NAC (1220 mg/kg), respectively. Mice were sacrificed at 0.5-hour and 1-hour post-dosing and ˜600 μL of blood per mouse were collected in heparinized centrifuge tubes. Blood was acidified with HNO₃ and subjected to ICP-MS for bismuth content measurement. Blood from untreated mice was collected and used as control to eliminate matrix effects. For the measurement of bismuth accumulation in mouse lung, groups of Balb/c mice (n=3 per group) were orally administered with CBS (150 mg/kg)+10NAC (610 mg/kg) for 1 day, consecutive 2 days, consecutive 3 days. The lung tissues were dissected after cardiac perfusion with 0.9% saline, and acidified with 69% HNO₃ for the measurement of bismuth content by using ICP-MS.

For the measurement of detailed pharmacokinetics profiles of the optimal NAC combination with CBS identified from the above mice study, rats received a minor surgery of cannulation one day prior to experiment, with a polythene tube (i.d.0.4 mm×o.d. 0.8 mm, Harvard Apparatus, USA) in the left jugular vein, followed by an overnight recovery and fasting. Two group of rats (n=5 per group) were orally administrated with 1-mL aliquot of CBS (150 mg/kg) or CBS (150 mg/kg)+10NAC (610 mg/kg). About 200 μL of rat blood were collected via the jugular vein cannula into a heparinized centrifuge tube at 0.17, 0.33, 0.5, 1, 2, 4, 6, 8, 12, 24-hour post drug administration for respective group. Rats were allowed for free access to food 12 h after drug administrations. All the rats were sacrificed 24-hour post dosing followed by cardiac perfusion with 200-mL saline to collect major organs including spleen, liver, lung, kidney and brain for further analyses. For the digestion of tissues, a modified protocol from US EPA 3050B (USEPA, 1996) was used. Briefly, approximate 0.2˜0.3 g of the respective rat organ samples was placed in 15 ml polypropylene tubes and digested with 1 mL of 69% HNO₃ at 65° C. for 16 h, while 100 μL of rat blood was digested with equal volume of 69% HNO₃ at 65° C. for 16 h. After being cooled down to room temperature, the samples were diluted to 1% nitric acid to the final volume of 3 mL for further use. To eliminate matrix effect, blood and organs were also collected from untreated rats and digested under identical condition serving as blank control. Standard solutions were prepared by diluting multielement calibration standard of bismuth in the respective blank control of organ and blood, respectively. The bismuth content in each organ or blood sample was then measured by ICP-MS and calculated according to the standard curve in respective blank control. Pharmacokinetic parameters including the peak concentration (Cₘₐₓ), the area under the concentration-time curve (AUC), the time reaching Cₘₐₓ (Tₘₐₓ) were determined through noncompartmental analysis with Phoenix WinNonlin version 6.4 (Pharsight Corporation, Mountain View, Calif., USA).

Nephrotoxicity Test

Groups of mice (n=4 per group) were orally administered with water as vehicle, CBS (500 mg/kg) and CBS (500 mg/kg)+3NAC (580 mg/kg) 4 consecutive days, respectively. Mice were sacrificed at 1, 7, 14, 28 day post last dosing and mice serum was collected for the blood urea nitrogen test (ThermoFisher, USA) and creatinine test (Cayman Chemical, Mich., USA) according to the manufacturer's instruction. Serum isolated from untreated mice were used as control.

Plaque Reduction Assay

Plaque reduction assay was performed to plot the 50% antiviral effective dose (EC₅₀) of individual compound as previously described with slight modifications⁴⁰. Briefly, VeroE6 cells were seeded at 4×10⁵ cells per well in 12-well tissue culture plates on the day before carrying out the assay. After 24 hr-incubation, 50 plaque-forming units (PFU) of SARS-CoV-2 were added to the cell monolayer in the presence or absence of drug compounds and the plates to be further incubated for 1 hr at 37° C. in 5% CO₂ before removal of unbound viral particles by aspiration of the media and washing once with DMEM. Monolayers were then overlaid with Dulbecco's modified eagle medium (DMEM) containing 1% low melting agarose (Cambrex Corporation, New Jersey, USA) and appropriate concentrations of individual compound, inverted and incubated as above for another 72 hrs. The wells were then fixed with 10% formaldehyde (BDH, Merck, Darmstadt, Germany) overnight. After removal of the agarose plugs, the monolayers were stained with 0.7% crystal violet (BDH, Merck) and the plaques were counted. The percentage of plaque inhibition relative to the control (i.e., without the addition of compound) wells were determined for each drug compound concentration. The EC50 were calculated using Sigma plot (SPSS) in an Excel add-in ED50V10. Selectivity index was calculated as the ratio of CC₅₀ over EC₅₀.

In studies using NAC, Plaque reduction assay was performed to estimate the half maximal effective concentration (EC50) as previously described with slight modifications(2, 15). Briefly, VeroE6 cells were seeded at 4×10⁵ cells/well in 12-well tissue culture plates on the day before the assay was performed. After 24 hour of incubation, 50 plaque-forming units (PFU) of SARS-CoV-2 were added to the cell monolayer with or without the addition of CBS, NAC or CBS+3NAC at varying concentrations. The plates were further incubated for 1 hour at 37° C. in 5% CO₂ before removal of unbound viral particles by aspiration of the media and washing once with DMEM. Monolayers were then overlaid with media containing 1% low melting agarose (Cambrex Corporation, New Jersey, USA) in DMEM and appropriate concentrations of trichostatin A, inverted and incubated as above for another 72 hours. The wells were then fixed with 10% formaldehyde (BDH, Merck, Darmstadt, Germany) overnight. After removal of the agarose plugs, the monolayers were stained with 0.7% crystal violet (BDH, Merck) and the plaques were counted. The percentage of plaque inhibition relative to the control (i.e. without the addition of compound) wells was determined for each concentration of drug compound. EC₅₀ was calculated using Sigma plot (SPSS) in an Excel add-in ED50V10. The plaque reduction assay experiments were performed in triplicate and repeated twice for confirmation.

Viral Load Reduction Assay

Viral load reduction assay was performed on VeroE6 and Caco2 cells, as described previously with modifications⁴¹. Supernatant samples from the infected cells (0.1 MOI) were harvested at 48 hours post-infection (hpi) for qRT-PCR analysis of virus replication. Briefly, 100 μL of viral supernatant were lyzed with 400 μL of AVL buffer and then extracted for total RNA with the QIAamp viral RNA mini kit (Qiagen, Hilden, Germany). Real-time one-step qRT-PCR was used for quantitation of SARS-CoV-2 viral load using the QuantiNova Probe RT-PCR kit (Qiagen) with a LightCycler 480 Real-Time PCR System (Roche) as previously described¹⁵. Each 20 reaction mixture contained 10 μL of 2×QuantiNova Probe RT-PCR Master Mix, 1.2 μL of RNase-free water, 0.2 μL of QuantiNova Probe RT-Mix, 1.6 μL each of 10 μM forward and reverse primer, 0.4 μL of 10 μM probe, and 5 μL of extracted RNA as the template. Reactions were incubated at 45° C. for 10 min for reverse transcription, 95° C. for 5 min for denaturation, followed by 45 cycles of 95° C. for 5 s and 55° C. for 30 s. Signal detection and measurement were taken in each cycle after the annealing step. The cycling profile ended with a cooling step at 40° C. for 30 s. The primers and probe sequences are against the RNA-dependent RNA polymerase/Helicase (RdRP/Hel) gene region of SARS-Cov-2 previously described⁴².

In other studies, SARS-CoV-2-infected (MOI=0.01) Vero E6 cells were treated with different concentrations of either CBS or CBS+3NAC. Cell culture supernatants were collected at 48 hour-post-infection (hpi) for viral RNA extraction and quantitative reverse transcription-polymerase chain reaction (qRT-PCR) as previously described with modifications(13, 14). The primers and probe sequences were against the RNA-dependent RNA polymerase/Helicase (RdRP/Hel) gene region of SARS-CoV-2: forward primer: 5′-CGCATACAGTCTTRCAGGCT-3′ (SEQ ID NO:6); reverse primer: 5′-GTGTGATGTTGAWATGACATGGTC-3′(SEQ ID NO:7); specific probe: 5′-FAMTTAAGATGTGGTGCTTGCATACGTAGAC-IABkFQ-3′(SEQ ID NO:8). The viral load reduction assay experiments were performed in triplicate and repeated twice for confirmation.

Time of Drug-Addition Assay

Time of drug-addition assay was performed to investigate which stage of SARS-CoV-2 life cycle the compound interfered with. Briefly, VeroE6 cells were seeded in 24-well plates (2×10⁵ cells/well). The cells were infected with SARS-CoV-2 (MOI=2) and then incubated for 1 h. The viral inoculum was then removed and the cells were washed twice with PBS. At 1 hpi (i.e., post-entry), the selected drugs at an appropriate concentration were added to the infected cells, followed by the incubation at 37° C. in 5% CO₂ until 9 hpi (i.e. one virus life cycle). For the time point of “−2 to 0 hpi” (i.e. pre-incubation), drugs were added at 2 h before SARS-CoV-2 inoculation and removed at 0 h, which was followed by virus inoculation as described above. For the time point “0-1 hpi” (i.e. co-infection), drugs were added together with the virus inoculation at 0 hpi, followed by drug removal at 1 hpi and incubated in the fresh medium until 9 hpi. Drug maintained full-course of the infection was taken as a positive control, whereas DMSO was included as a negative control in each of four treatments. At 9 hpi, the cell culture supernatant of each time point experiment was collected for viral yield measurement using qRT-PCR as described above.

For experiments using CBS+3NAC, Vero E6 cells were seeded in 96-well plates (4×10⁴ cells per well). The cells were infected by SARS-CoV-2 HKU-001a at an MOI of 1.5 and then incubated for additional 1 hour. The viral inoculum was then removed, and the cells were washed twice with PBS. At 1 hour after inoculation (that is, after entry), CBS+3NAC at a concentration of 1000 μM was added to the infected cells at time points indicated, followed by incubation at 37° C. in 5% CO₂ until 10 hours after inoculation (that is, one complete virus life cycle). Cells were fixed at 10 hours after inoculation for the quantification of the percentage of infected cells using an immunofluorescence assay targeting SARS-CoV-2 NP.

Protein Purification

The gene cloning and protein purification were performed according to previously described method^{(17, 18)}. Genes encoding SARS-CoV-2 papain-like protease) (PL^{pro}) (ORFlab polyprotein residues 1564-1882), main protease (M^{pro}) (ORFlab polyprotein residues 3264-3569) were cloned into the expression vector pETH, respectively, and Genes encoding SARS-CoV-2 helicase (Hel) (ORFlab polyprotein residues 16237-18039) was cloned into the expression vector pET28-a(+). The recombinant proteins were overexpressed in E. coli BL21(DE3) and purified using the Ni²⁺-loaded HiTrap Chelating System (GE Healthcare) according to the manufacturer's instructions. The product was further purified by gel filtration using a HiLoad 16/600 Superdex 200 prep grade column (GE Life Sciences). The purity of each protein was assessed by 12% sodium dodecyl sulfate-polyacrylamide gels (SDS-PAGE). Apo-SARS-CoV-2 PL^{pro} (20 μM) was prepared by dialysis in Zn²⁺ chelating buffer (20 mM Tris-HCl, pH 8.0, 150 mM NaCl, 2 mM EDTA, 2 mM 4 TCEP, 20% glycerol) and removal of excess EDTA by ultrafiltration (Amicon). The concentration of each protein was determined by using the Bicinchoninic Acid Protein Assay Kit (Sigma-Aldrich).

Immunofluorescence Microscopy

Vero E6 cells were infected with SARS-CoV-2 (MOI=0.1) and exposed to the treatment of water as vehicle, CBS (1000 μM), NAC (1000 μM), and CBS (1000 μM)+3NAC (3000 μM), respectively, for 24 hour. Antigen expression in SARS-CoV-2-infected cells were detected with an in-house rabbit antiserum against SARS-CoV-2-N nucleocapsid protein (NP). Cell nuclei were labelled with the 4,6-diamidino-2-phenylindole (DAPI) nucleic acid stain from Thermo Fisher Scientific (Waltham, Mass., USA). The Alexa Fluor secondary antibody were obtained from Thermo Fisher Scientific. Mounting was performed with the Diamond Prolong Antifade mountant from Thermo Fisher Scientific.

Anti-SARS-CoV-2 Evaluation of the Selected Compound in Golden Syrian Hamster Model

Male and female Syrian hamster, aged 6-10 weeks old, were kept in biosafety level housing and given access to standard pellet feed and water ad libitum as previously described¹⁹. All experimental protocols were approved by the Animal Ethics Committee in the University of Hong Kong (CULATR) and were performed according to the standard operating procedures of the biosafety level 3 animal facilities (Reference code: CULATR 5370-20).

Experimentally, each hamster was intranasally inoculated with 10⁴ PFU of SARS-CoV-2 in 100 μL PBS under intraperitoneal ketamine (200 mg/kg) and xylazine (10 mg/kg) anaesthesia. Six-hours post-virus-challenge, hamsters were intraperitoneally given either Pylorid (150 mg/kg/day), or remdesivir (25 mg/kg/day) or PBS (untreated controls) for consecutive four days.

Animals were monitored twice daily for clinical signs of disease. Their body weight and survival were monitored for 14 dpi. Five animals in each group were sacrificed at 4 dpi for virological and histolopathological analyses. Lung and nasal turbinate tissue samples were collected. Viral yield in the tissue homogenates were detected by TCID₅₀ and qRT-PCR method, respectively. Cytokine and chemokine profile of the hamster lungs were detected by 2^{-ΔΔCT} method using probe-based one-step qRT-PCR (Qiagen). Probe and primer sequences for each gene detection were listed in Table 3.

TABLE 3
Probe and primer sequences

| | Forward | Reverse | Probe |
|---|---|---|---|
| Genes | (5′ to 3′) | (5′ to 3′) | (5′ to 3′) |
| Hamster | TGA GCC ATC | AGC CCG TCT | (6FAM)-CGG |
| TNF-α | GTG CCA ATG | GCT GGT ATC | CAT GTC TCT |
| | (SEQ ID NO: | AC (SEQ ID | CAA AGA CAA |
| | 9 | NO: 10) | CCA CCA G- |
| Hamster | TGT TGC TCT | AAG ACG AGG | (6FAM)-TGG |
| IFN-γ | GCC TCA CTC | TCC CCT CCA | CTG CTA CTG |
| | AGG (SEQ ID | TTC (SEQ ID | CCA GGG CAC |
| | NO: 12) | NO: 13) | ACT C- |
| | | | (TAMRA) |
| | | | (SEQ ID |
| | | | NO: 14) |
| Hamster | TGT CTT GGG | CCA AAC CTC | — |
| IL-6* | ACT GCT GC | CGA CTT GTT | |
| | (SEQ ID NO: | GA (SEQ ID | |
| | 15) | NO: 16) | |
| Hamster | GGT TGC CAA | TTC ACC TGT | (6FAM)-TGC |
| IL-10 | ACC TTA TCA | TCC ACA GCC | AGC GCT GTC |
| | GAA ATG | TTG (SEQ ID | ATC GAT TTC |
| | (SEQ ID NO: | NO: 18) | TCC C- |
| | 17) | | (TAMRA) |
| | | | (SEQ ID |
| | | | NO: 19) |
| Hamster | TGG TGC CAA | GAA CTC CTT | (6FAM)-CTG |
| CCL22 | CGT GGA AGA | CAC TAC GCG | CCA GGA CTA |
| | C (SEQ ID | C (SEQ ID | CAT CCG TCA |
| | NO: 20) | NO: 21) | GC-(TAMRA) |
| | | | (SEQ ID |
| | | | NO: 22) |
| Hamster | GCT TGG TCA | GTG GTT GCG | (6FAM)-TCC |
| CCR4 | CGT GGT CAG | CTC CGT GTA | CTC CCA GGC |
| | TG (SEQ | G (SEQ ID | CTC TTG AGC- |
| | ID NO: 23) | NO: 24) | (TAMRA) (SEQ |
| | | | ID NO: 25) |
| Hamster | ACA GAG AGA | GCC TGA ATG | (VIC)-TTG AAA |
| γ-actin | AGA TGA CGC | GCC ACG TAC | CCT TCA ACA |
| | AGA TAA TG | A (SEQ ID | CCC CAG CC- |
| | (SEQ ID NO: | NO: 27) | (TAMRA) (SEQ |
| | 26) | | ID NO: 28) |

*SYBR Green-based detection without using probe

Tissue pathology of infected animals were examined by H&E staining and immunofluorescence staining in accordance to the established protocol³⁸.

To differentiate lung pathology, semi-quantitative histology scores were given to lung tissue by grading the severity of damage in the bronchioles, alveoli and blood vessels and accumulating the total scores as follows. Bronchioles: 0, normal structure; 1, mild peribronchiolar infiltration; 2, peribronchiolar infiltration plus epithelial cell death; 3, score 2 and intra-bronchiolar wall infiltration and epithelium desquamation. Alveoli: 0, normal structure; 1, alveolar wall thickening and congestion; 2, focal alveolar space infiltration or exudation; 3, diffuse alveolar space infiltration or exudation or hemorrhage. Blood vessel: 0, normal structure; 1, mild perivascular oedema or infiltration; 2, vessel wall infiltration; 3, severe endothelium infiltration.

For NAC studies, Each hamster was intranasally inoculated with 10⁵ p.f.u. of SARS-CoV-2 (SARS-CoV-2 HKU-001a) in 100 μL PBS under intraperitoneal ketamine (200 mg per kg body weight) and xylazine (10 mg per kg body weight) anesthesia. From −3 day-post-infection (dpi) to 1 dpi, hamsters were orally administered once daily with water as vehicle, CBS, NAC and CBS+3NAC, respectively, for four consecutive days. Animals were monitored twice daily for clinical signs of disease. Eight animals in each group were euthanized at 2 dpi. for virological and histopathological analyses. Lung tissue samples were isolated. Viral yield in the tissue homogenates was detected by qRT—PCR methods. The cytokine and chemokine profiles of the hamster lungs were detected by the 2^{-ΔΔCT} method using probe-based one-step qRT-PCR (Qiagen). The tissue pathology of infected animals was examined by H&E and immunofluorescence staining in accordance with an established protocol (16).

Gene Cloning and Construction of Plasmid for SARS-CoV-2 Helicase

SARS-CoV-2 helicase (i.e., nsp13) is one of the cleavage products (non-structural proteins) of the viral polyprotein ORFlab. The coding sequence of nsp13 is within the range from 16237 to 18039 of the Severe acute respiratory syndrome coronaviruses 2 isolate Wuhan-Hu-1, complete genome (NCBI GenBank accession No.: NC_045512.2). There is neither start nor stop codon. The DNA fragment of full-length nsp13 was amplified from the viral cDNA using the primer pair of nCoV-nsp-13-F (BamHI):

| (SEQ ID NO: 1) | |
|---|---|
| | CGGGATCCATGGCTGTTGGGGCTTGTGTTCTT |

and nCOV-nsp-13-R (XhoI):

| (SEQ ID NO: 2) | |
|---|---|
| | CCGCTCGAGTCATTGTAAAGTTGCCACATTCCTAC |

by Phusion® High-Fidelity DNA Polymerase (New England Biolabs) in a Veriti™ 96-Well Thermal Cycler (Applied Biosystems). The cDNA was synthesised from viral RNA by transcriptor first strand cDNA synthesis kit (Roche) using random hexamer primers. The thermocycling conditions: 98° C. for 30 s (initial denaturation), followed by 30 cycles of amplification (98° C. 30s, 62° C. 10 s and 72° C. 2min). The PCR product was digested by BamHI and XhoI, and then inserted into the plasmid pET28-a(+) using T4 DNA ligase, generating pET28-nsp13, which produces His₆- and T7-tagged helicase. The ligation product was transformed into Escherichia coli (E. coli) DH10B prior to sequencing validation. The validated plasmid pET28-nsp13 was then transformed into E. coli BL21(DE3).

Overexpression and Purification of SARS-CoV-2 Helicase

E. coli BL21(DE3) harbouring pET28-nsp13 was cultured in LB medium overnight at 37° C. with supplementation of 50 μg/mL kanamycin. The culture was then amplified with 1:100 dilution factor in 1 L of LB medium until the OD₆₀₀ reached 0.6. The overexpression of the helicase was induced by 200 mM of IPTG at 25° C. for 16 hrs with agitation at 200 rpm. After overexpression, the bacterial pellet was collected by centrifugation at 5,000 g, 4° C. for 10 min and washed once with the binding Buffer A [20 mM Tris-HCl, pH6.8, 500 mM NaCl, 20 mM imidazole. The pellet was resuspended in buffer A supplemented with 0.1% Triton X-100 and cOmplete™ Protease Inhibitor Cocktail (Roche) and lysed by sonication. The bacterial lysate was centrifuged at 13,000 g at 4° C. for 30 min. The supernatant was loaded on to buffer A-balanced a 5-mL Ni(II)-charged HiTrap® Chelating column (GE Life Sciences), which was then washed with Buffer A. The recombinant protein was eluted with the linear gradient of elution Buffer B [buffer A with 250 mM imidazole]. The collected fractions of eluent were checked using SDS-PAGE and purest fractions were pooled together and subjected for thrombin-digestion of His6-tag. The digested protein solution was loaded on to the 5-mL HiTrap® Chelating HP column to remove His6-tag and undigested protein. The product was further purified by gel-filtration using HiLoad® 16/600 Superdex® 200 prep grade column (GE Life Sciences) with gel-filtration Buffer C [20 mM Tris-HCl, pH 6.8, 250 mM NaCl]. An AKTA FPLC system (GE Life Sciences) was used for protein purification.

ATPase Assay

ATPase assays were performed by measuring the release of phosphate based on a modified method as previously described¹⁶ using ATPase Assay Kit (ab234055, Abcam, Cambridge, Mass., USA). In a volume of 48 μL, typically 2 nM protein was incubated with varying concentrations of Pylorid in ATPase reaction buffer [20 mM Tris-HCl, pH 7.4, 5 mM MgCl₂, 2 mM tris(2-carboxyethyl)phosphine (TCEP)] for 5 min at 25° C., followed by the addition of 1 μL of 100 mM ATP and 1 μL of 2 mg ml⁻¹ poly(U)to initiate the reaction. About 15 μL of the reaction developer were added to each well and the color was developed for 15 min. The mixtures were subsequently subjected to absorbance measurement at 650 nm using a SpectraMax® iD3 Multi-Mode microplate reader. The relative ATPase activity was the ratio between the activity of the samples in the presence of Pylorid and the activity of the control sample, and therefore expressed as a percentage. The assays were performed in triplicate and repeated on different days.

FRET-Based DNA Duplex Unwinding Assays

The FRET-based assays were performed based on a modified method as previously described¹⁶. DNA oligomers were synthesized and purified by HPLC: FL-Cy3 oligo (5′-TTTTTTTTTTTTTTTTTTTTCGAGCACCGCTGCGGCTGCACC(Cy3)-3′) (SEQ ID NO:3), RL-BHQ oligo (5′-(BHQ2)GGTGCAGCCGCAGCGGTGCTCG-3′) (SEQ ID NO:4) and RL oligo (5′-GGTGCAGCCGCAGCGGTGCTCG-3′) (SEQ ID NO:5) and RL oligo (5′-GGTGCAGCCGCAGCGGTGCTCG-3′) (Metabion GmbH, Germany) (18). The two oligomers were mixed in a ratio of FL-Cy3:RL-BHQ of 1:1.5 at the final concentrations of 10 μM and 15 μM, respectively, in annealing buffer [20 mM Tris-HCl pH 8.0, 150 mM NaCl] and annealed by heating to 90° C. for 2 min in thermocycler (S1000™ Thermal Cycler, Bio-Rad), then cooling slowly to 25° C. at the rate of ˜1° C./min. The FRET assay was performed by incubating 10 nM protein with varying concentrations of Pylorid in a 48.5 μL of helicase reaction mix [20 mM Tris-HCl buffer, pH 7.4, 150 mM NaCl, 0.1 mg/mL BSA, 5 mM MgCl₂, 5 mM TCEP, 5% glycerol] in 96-well black polystyrene microplate (Corning®) at 25° C., and then 0.5 uL of 100 mM ATP and 1.5 uL of oligo mixture was added to make the final concentration of FL-Cy3:RL-BHQ oligo and RL oligo at 5 nM and 10 nM, respectively. Reactions were incubated for 2 mins, and then the change in fluorescence (λₑₓ=550 nm, λₑₘ=620 nm) was measured using SpectraMax® iD3 Multi-Mode microplate reader to determine the extent of DNA duplex unwinding. The relative DNA unwinding activity was the ratio between the activity of the samples in the presence of Pylorid and the activity of the control sample, and therefore expressed as a percentage. The assays were performed in triplicate and repeated on different days.

For experiments using NAC, SARS-CoV-2 Hel (10 nM) was incubated with varying concentrations of CBS and CBS+3NAC in reaction buffer (20 mM Tris-HCl buffer, pH 7.4, 10 mM NaCl, 0.1 mg mL⁻¹ bovine serum albumin (BSA), 5 mM MgCl₂, 0.5 mM tris(2-carboxyethyl)phosphine (TCEP), 5% glycerol) in a 96-well black polystyrene microplate (Corning) at room temperature, then 0.5 μL of 100 mM ATP and 1.5 μL of oligo mixture were added to achieve final concentrations of FL-Cy3:RL-BHQ oligo and RL oligo of 5 nM and 10 nM, respectively. Fluorescence (λₑₓ=550 nm, λₑₘ=620 nm) was detected to determine DNA-duplex unwinding. The relative dsDNA unwinding activity was the ratio between the activity of the samples in the presence of bismuth drug and the activity of the control, and is therefore expressed as a percentage. The assay was performed in triplicate.

For ATPase activity inhibition in experiments using NAC, a colorimetric assay was performed by measuring the release of phosphate using on a previously described method (18) with an ATPase assay kit (ab234055, Abeam). Typically, SARS-CoV-2 Hel (2 nM) was incubated with varying concentrations of CBS and CBS+3NAC in reaction buffer (20 mM Tris-HCl, pH 6.8, 10 mM NaCl, 5 mM MgCl₂, 0.5 mM TCEP, 5% glycerol) for 30 min at room temperature, followed by the addition of ATP (2 mM) and poly(U) (0.4 mg mL⁻¹) to initiate the reaction. To a 50 μL reaction system added 15 μl of the reaction developer and the color was developed for 15 min. Absorbance was measured at 650 nm to determine ATPase activity. The relative ATPase activity was the ratio between the activity of the samples in the presence of bismuth drug and the activity of the control, and is therefore expressed as a percentage. The assay was performed in triplicate.

For PL^{pro} activity inhibition assay, a FRET-based assay was performed based on a previously described method with a peptide substrate Arg-Leu-Arg-Gly-Gly↓-AMC (RLRGG↓-AMC, Bachem Bioscience) (SEQ ID NO: 30) (19). SARS-CoV-2 PL^{pro} (50 nM) was incubated with CBS and CBS+3NAC at varying concentrations, respectively, for 90 min in reaction buffer (50 mM HEPES, pH 7.4, 10 mM NaCl, 0.1 mg/ml BSA, 5% glycerol, 0.5 mM TCEP) at room temperature, followed by the addition of RLRGG↓-AMC (2 μM) to initiate the reaction. After another 30 min incubation, fluorescence (λₑₓ=335 nm, λₑₘ=460 nm) was measured to determine PL^{pro} activity. The relative PL^{pro} activity was the ratio between the activity of the samples in the presence of bismuth drug and the activity of the control, and is therefore expressed as a percentage. The assay was performed in triplicate.

For M^{pro} activity inhibition assay, a FRET-based assay was performed based on a previously described method with a peptide substrate Dabcyl-KTSAVLQ←SGFRKM-E (SEQ ID NO:29)(Edans)-NH₂ (GL Biochem) (17, 20). SARS-CoV-2 M^{pro} (0.5 μM) was incubated with CBS and CBS+3NAC at varying concentrations, respectively, for 30 min in reaction buffer (20 mM Tris-HCl, pH 7.4, 20 1 mM NaCl, 0.1 mg/ml BSA, 5% glycerol, 0.5 mM TCEP) at room temperature, followed by the addition of Dabcyl-KTSAVLQ←SGFRKM-E (SEQ ID NO:29) (20 μM) to initiate the reaction. After another 30 min incubation, fluorescence (λₑₓ=335 nm, λₑₘ=460 nm) was measured to determine M^{pro} activity. The relative M^{pro} activity was the ratio between the activity of the samples in the presence of bismuth drug and the activity of the control, and is therefore expressed as a percentage. The assay was performed in triplicate.

Reaction Kinetics of Bi³⁺ with Proteins

Kinetics of reaction of Bi³⁺ with PL^{pro} and M^{pro} were performed by UV-vis spectrophotometry. Briefly, proteins (PL^{pro}: 20 μM and M^{pro}: 30 μM) were firstly prepared in reaction buffer (20 mM Tris-HCl, pH 7.4, 10 mM NaCl, 5% glycerol, 0.2 mM TCEP) in a 96-well UV-transparent microplates (Corning®) and then incubated with 30 mol eq. CBS at room temperature. The absorbance was recorded for 20 hours at 340 nm, 25° C. to monitor the equilibrium situation in a kinetics mode using a SpectraMax iD3 multimode microplate reader. The kinetic data were analyzed by a nonlinear square fitting based on a one-phase exponential function using Prism 8.0 (GraphPad Software Inc.) software.

Michaelis-Menten Kinetics

For ATPase assays in experiments using pylorid, SARS-CoV-2 helicase (0.5 nM) was incubated with Pylorid (0, 0.01, 0.05, 0.1 and 0.5 μM) in ATPase reaction mixture in a total volume of 50 μL, respectively, at 25° C. for 30 mins. To each aliquot of reaction mix was added 30 μL of the reaction developer and then ATP as substrate make the final concentrations of 0.2, 0.5, 1, 2, 4, 6, 8 mM. Control experiment was performed in the absence of inhibitors under the same conditions. The values of Vₘₐₓ, Kₘ and Ki for both uninhibited and inhibited reactions were obtained by fitting the data into the double reciprocal Lineweaver-Burk plots. For DNA unwinding assays, SARS-CoV-2 helicase (10 nM) was incubated with Pylorid (0, 0.1, 0.2, and 0.5 μM) in helicase reaction mix at 25° C. for 5 mins. FL-Cy3:RL-BHQ oligo and RL oligo were added to the enzyme to make the final substrate concentrations of 2.5, 5, 7.5, 10, 15, and 20 nM. The control experiment was performed in the absence of inhibitors under the same conditions. The values of Vₘₐₓ, Kₘ and Ki for both uninhibited and inhibited reactions were obtained by fitting the data into the double reciprocal Lineweaver-Burk plots.

For PL^{pro} assay, reaction mix was prepared by incubating SARS-CoV-2 PL^{pro} (20 nM) with CBS+3NAC (0, 0.1, 0.5, 1 and 2 mM) in the reaction buffer (50 mM HEPES, pH 7.4, 10 mM NaCl, 0.1 mg/ml BSA, 5% glycerol, 0.5 mM TCEP) in a total volume of 100 μL at room temperature for 4 hours. To each aliquot of reaction mix, substrate RLRGG↓-AMC was added to achieve final concentrations of 0.5, 1, 2, 5, 10, 20 μM. The control experiment was performed in the absence of inhibitors under the same conditions.

For M^{pro} assay, reaction mix was prepared by incubating SARS-CoV-2 PL^{pro} (0.5 μM) with CBS+3NAC (0, 2, 10, 20 μM) in the reaction buffer (20 mM Tris-HCl, pH 7.4, 10 mM NaCl, 0.1 mg/ml BSA, 5% glycerol, 0.5 mM TCEP) in a total volume of 100 μL at room temperature for 4 hours. To each aliquot of reaction mix, substrate Dabcyl-KTSAVLQ←SGFRKM-E was added to achieve final concentrations of 10, 25, 50, 75, 100, 150, 200 μM. The control experiment was performed in the absence of inhibitors under the same conditions. The values of Vₘₐₓ, Kₘ and Kᵢ for both uninhibited and inhibited reactions were obtained by fitting the data into the double reciprocal Lineweaver-Burk plots.

Zinc Supplementation Assay

Apo-SARS-CoV-2 helicase (10 μM) was firstly prepared by dialysis in zinc(II) chelating buffer [20 mM Tris-HCl, pH 8.0, 150 mM NaCl, 0.5 mM EDTA, 2 mM TCEP]. Bismuth bound SARS-CoV-2 helicase was then prepared by dialyzing apo-proteins with excess amounts of bismuth(III) nitrate in glycerol dialysis buffer [50 mM Tris-HCl, pH 7.4, 20 mM NaCl, 5 mM TCEP, 20% glycerol] at 4° C. overnight, followed by removal of unbound Bi(III) and verification of the bound Bi(III) by ICP-MS. The resulting protein was mixed with ZnSO₄ at concentrations up to 50 molar equivalents for SARS-CoV-2 and incubated for 2 hrs at room temperature and then subjected for the ATPase and DNA unwinding assays as mentioned above.

UV-vis Spectroscopy V-vis spectroscopic titration was carried out on a Varian Cary 50 spectrophotometer at a rate of 360 nm/min using a 1-cm quartz cuvette at 25° C. Aliquots of 2 mM Bi³⁺ (as Bi(NTA)₃) stock solution were stepwise titrated into apo-SARS-CoV-2 helicase (10 μM) in a titration buffer [50 mM Tris-HCl, pH 7.4, 20 mM NaCl, 2 mM TCEP]; aliquots of 2 mM Bi³⁺ (as Bi(NTA)₃) stock solution were stepwise titrated into proteins (apo-SARS-CoV-2 PL^{pro}: 10 μM, M^{pro} 20 μM) in titration buffer (20 mM Tris-HCl, pH 7.4, 10 mM NaCl, 1 mM TCEP). UV-vis spectra were recorded in a range of 250-600 nm at appropriate time interval between each addition and in some experiments, 10 min after each addition. The binding of bismuth(III) to the protein was monitored by the increase in absorption at ˜340 nm. The UV titration curve was fitted with Ryan-Weber nonlinear equation and K_{d} was estimated.

Zinc Displacement Analysis

SARS-CoV-2 helicase (3 μM) was incubated with 10 μM ZnSO₄ in dialysis buffer [20 mM Tris-HCl, pH 7.4, 150 mM NaCl, 5 mM TCEP] overnight at 4° C., and the unbound Zn(II) ions were removed by dialysis in Zn free dialysis buffer to ensure that Zn(II) was fully loaded into the proteins. The resulting protein was then incubated with various concentrations of Pylorid by dialysis at 4° C. overnight with mild shaking. The samples were subsequently dialyzed in the dialysis buffer to remove unbound-metal ions, and were then acidified by concentrated HNO₃ at 60° C. for 2 hrs. Samples were diluted to a detectable concentration range and subjected to ICP-MS analysis (Agilent 7700x, Agilent Technologies, Calif., USA) with ¹¹⁵In as an internal standard for ²⁰⁹Bi, ⁶⁶Zn. Protein concentrations were quantified by standard bicinchoninic acid (BCA) assay (Thermal Fisher Scientific, USA).

### Zinc Release Assays.

The release of Zn²⁺ from SARS-CoV-2 PL^{pro} upon bismuth drug exposure was perform by a previously described method with zinc-specific fluorophore FluoZin™-3 (Invitrogen/Life Technologies) (21). Briefly, SARS-CoV-2 PLpro (20 μM) was incubated with 0, 2, 5, 20 mol eq. Bi³⁺ (as CBS), for 180 min at room temperature, followed by the addition of FluoZin-3 (1 μM) in a total reaction volume of 100 μL (50 mM Tris-HCl, pH7.4) at room temperature. Fluorescence (λₑₓ=494 nm, λₑₘ=530 nm) was detected and converted into Zn²⁺ concentrations using standard curves prepared from Zn(SO₄)₂ under identical condition. The signals of CBS at corresponding concentrations after mixing with FluoZin™-3 were recorded for background subtraction. The assay was performed in triplicate and the results were plotted as the [Zn²⁺]/[PL^{pro}] verse [Bi³⁺]/[PL^{pro}].

Ellman's Assay

Amount of free cysteine was assayed spectrophotometrically with DTNB [5,5′-dithiobis-(2-nitrobenzoic acid)]according to a previously described method⁽²²⁾. Briefly, SARS-CoV-2 M^{pro} (15 μM) was incubated with 0, 2, 5, 20 mol eq. Bi³⁺ (as CBS) in a 50 μL reaction buffer (20 mM Tris-HCl, pH 7.4, 10 mM NaCl) in a 96-well microplate for 60 min at room temperature. Equal volume of DTNB (2 mM) was added to the reaction mix in a total reaction volume of 100 μL. After the incubation for 90 min, absorption of 412 nm which indicated the release of 5-thiobenzoate anion, was detected and converted into thiol concentrations using standard curves prepared with reduced glutathione (GSH) under identical condition. The assay was performed in triplicate and the results were plotted as the [free cysteine]/[M^{pro}] verse [Bi³⁺]/[M^{pro}].

Statistical Analysis

All statistical analyses were performed on three independent experiments, or more if otherwise stated, using Prism 8.0 (GraphPad Software Inc.) software.

Results

The Selected Metallo-Compounds Exhibit Potent Activity Against SARS-CoV-2 In Vitro

Metal compounds are historically used as antimicrobial agents; however, their antiviral activities have not been explored extensively. In this study six metal compounds were selected, including two bismuth(III) citrate-based drugs, i.e., Colloidal Bismuth Subcitrate (CBS, De-Nol) and Ranitidine Bismuth Citrate (Pylorid, Jindele), two bismuth(III) porphyrins, i.e., Bi(TPP) (TPP: tetraphenylporphyrinate) and Bi(TPyP) (TPyP: tetra(4-pyridyl)porphyrin), one Au(I)-based drug Auranofin (Ridaura) as well as its cellular active form chloro(triethylphosphine)gold(I) (Au(PEt₃)Cl), for a primary screening against SARS-CoV-2 in vitro. The 50% cytotoxicity concentrations (CC₅₀) of these compounds in the monkey kidney VeroE6 cells were determined as 3254±21 μM μM for De-Nol, 2243±43 μM for Pylorid, >400 μM for both Bi(TPP) and Bi(TPyP), 14.2±1.3 μM for Ridaura and 13.5±1.8 μM for Au(PEt₃)Cl (Table 1).

Data TABLE 1
Cytotoxicity and antiviral activity of the selected compounds.
Summary of CC₅₀ and EC₅₀ of the selected compounds

| | CC₅₀ (μM) | | EC₅₀ (μM) | Selectivity index |
|---|---|---|---|---|
| Compound* | VeroE6 | Caco2 | VeroE6 | VeroE6 |
| De-Nol | 3254±21 | 3740±125 | 4.6±0.4 | 707 |
| Pylorid | 2243±43 | 2486±654 | 2.3±0.5 | 975 |
| Bi(TPP) | >400^{#} | >400 | 3.9±1.2 | >103 |
| Bi(TPyP) | >400 | >400 | 7.5 | >53 |
| Ridaura | 14.2±1.3 | N.D^{&} | N.D | N.D |
| Au(PEt₃)Cl | 13.5±1.8 | N.D | N.D | N.D |

^{&}N.D: not determined.
^{#}indicates the maximal soluble concentration used in the study.
*The measure of drug concentrations was based on metal content

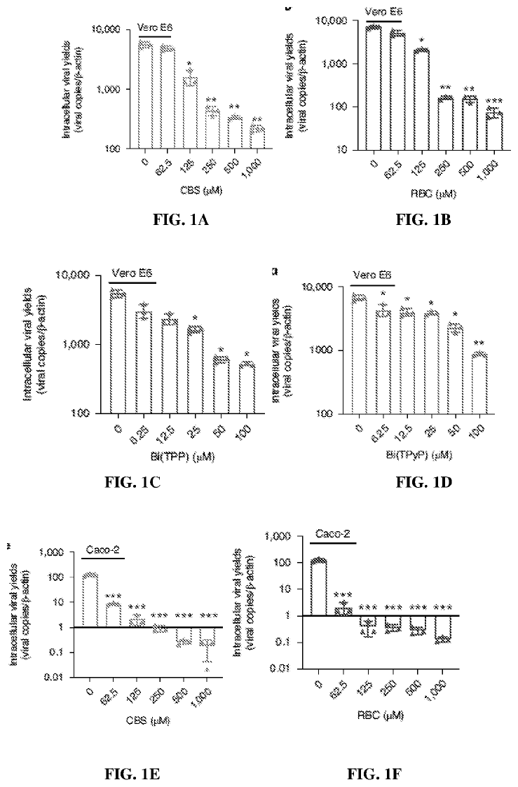

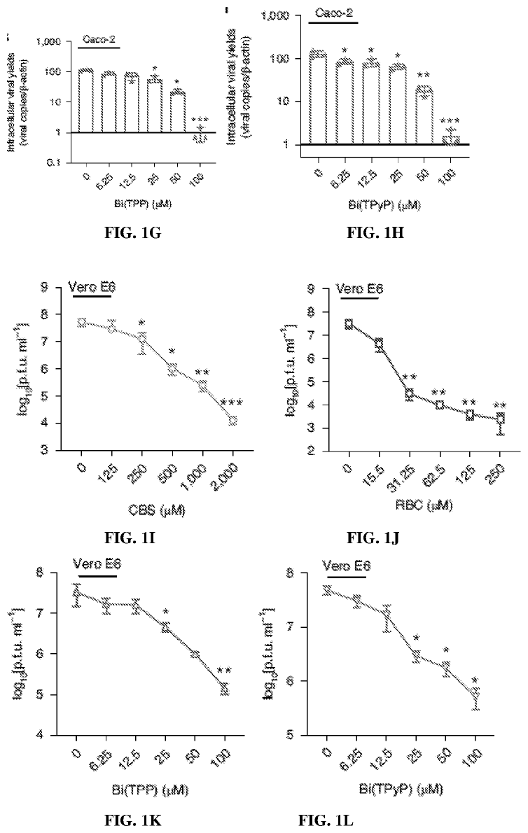

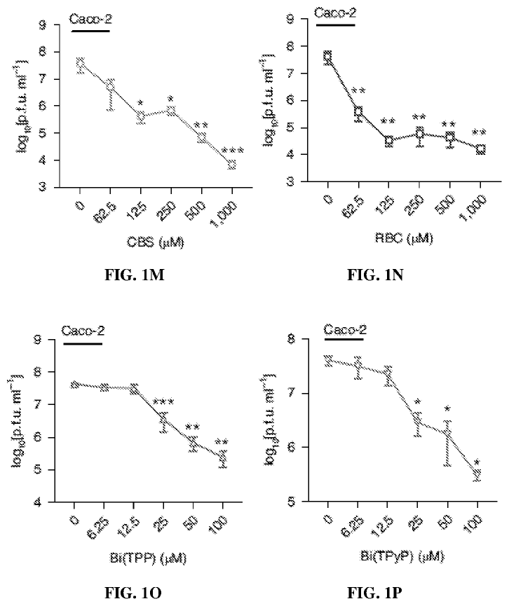

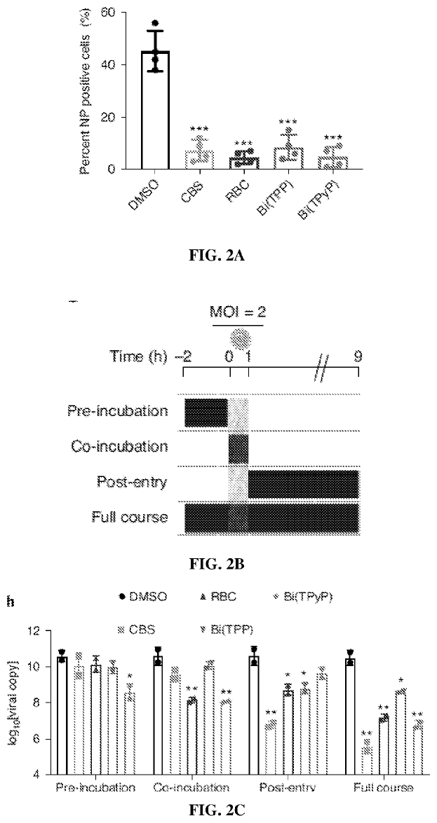

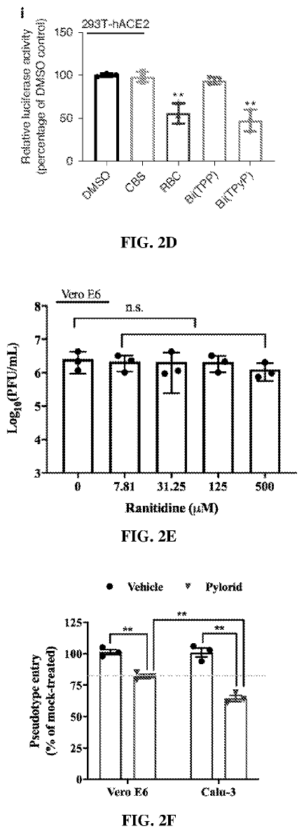

Next, the four bismuth(III) compounds were prioritized for further evaluation of their CC₅₀ values in human colorectal Caco2 cells due to their promisingly low cytotoxicity when compared with the Au(I)-based drugs, resulting with similar CC₅₀ values that range from 400 to 3740 μM, individually (Table 1). To evaluate their antiviral potency, the half maximal effective doses (EC₅₀) of bismuth(III) compounds were determined at low micro molar level and as 4.6±0.4 μM for De-Nol, 2.3+0.5 μM for Pylorid, 3.9±1.2 μM for Bi(TPP) and 7.5+0.9 μM for Bi(TPyP). Remarkably, addition of all the four bismuth(III) compounds at 1 hour post-infection (hpi) reduced viral RNA loads in both VeroE6 and Caco2 cells in a dose-dependent manner (FIG. 1A-Q). Under non-toxic concentrations, De-Nol and Pylorid exhibited more potent anti-SARS-CoV-2 activity than Bi(TPP) and Bi(TPyP), which was evidenced by the maximal ˜2 logs vs. 1 log viral load reduction in the VeroE6 cell lysate (FIG. 1A-1D); ˜3 logs vs. ˜2 logs reduction in the Caco2 cell lysate (FIG. 1E-1H); ˜4 logs vs. ˜3 logs reduction in VeroE6 cell culture supernatant (FIG. 1I-1L); and ˜4 logs vs. ˜3 logs reduction in Caco2 cell culture supernatant (FIG. 1M-1P). Bismuth(III) drugs/compounds greatly inhibited SARS-CoV-2 as evidenced by the markedly decreased expression of viral nucleoprotein in the drug-treated cells when compared with the DMSO-treated group (data not shown, and FIG. 2A). Collectively, the data demonstrate that bismuth(III) drugs/compounds robustly inhibit SARS-CoV-2 replication in vitro.

To investigate which steps of the SARS-CoV-2 replication cycle were interrupted by the selected drug compounds, a time-of-drug-addition assay was performed by treating virus-infected cells with each compound at different time points, followed by measurements of viral titer after 9 hpi, when the first round of progeny virions was detectable in the cell culture supernatant. (FIGS. 2B and 2C). Intriguingly, addition of Bi(TPyP) during cells pre-treatment or virus co-infection significantly suppressed virus replication, whereas no detectable effect was found when Bi(TPyP) was maintained after virus entry, indicating that Bi(TPyP) may interfere with SARS-CoV-2 attachment to the cellular surface. Pylorid did not affect virus replication when added during the pre-incubation stage, while it reduced viral loads for ˜2 logs when added during virus absorption and post-entry stages, suggesting that Pylorid is a multiple-target drug that acts during virus entry/internalization or early events after viral entry. To validate this, the interruption of virus entry by RBC and Bi(TPyP) was confirmed by a pseudotyped virus infection assay, showing that the percent virus entry was lowered by ˜45% and 53% by RBC and Bi(TPyP), respectively. (FIG. 2D. Apparently, both De-Nol and Bi(TPP) functioned at post-entry stages. Collectively, the vulnerability SARS-CoV-2 upon treatment with bismuth(III)-based drugs was demonstrated.

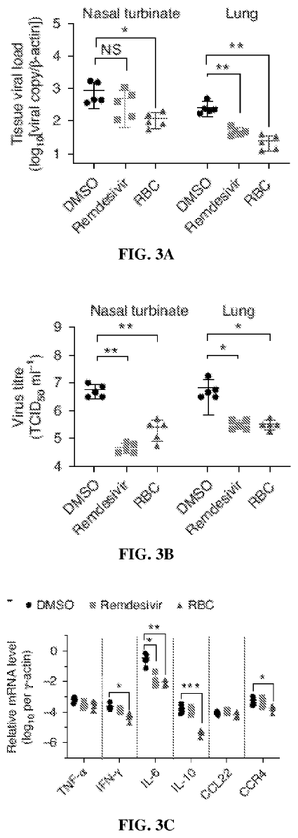

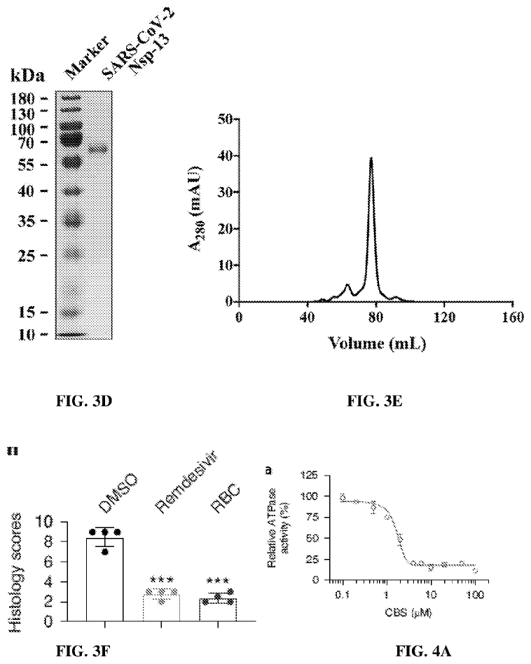

Therapeutic treatment with Pylorid mitigates SARS-CoV-2 disease Given the extraordinarily high selectivity index (975) of Pylorid against SARS-CoV-2, as well as the exclusive role of ranitidine alone, one of the two components of Pylorid, for virus inhibition (FIG. 2E), Pylorid was prioritized for in vivo antiviral evaluation. Pylorid is a clinically used drug for the treatment of Helicobacter pylori infection and gastric ulcer, whose safety profile in terms of human usage is well-documented¹⁷. Previous pharmacokinetics study revealed that Pylorid has a relatively rapid absorption (tₘₐₓ(bismuth) ˜0.5 h, tₘₐₓ(ranitidine) ˜2.5 h) and small renal clearance (CL_{T} (bismuth) ˜40 mL/min) with pharmacokinetic behavior linear within the dose range through intragastric administration¹⁸. Recent studies established an in vivo model to simulate the clinical and pathological manifestations of COVID-19 in golden Syrian hamsters, an excellent tool to study disease pathogenesis, transmissibility and antiviral evaluation¹⁹. In a pilot study, intraperitoneal injection of 150 mg/kg/day of Pylorid exhibited no significant toxicity to the animals. Remdesivir was included as a positive control drug and dosed at 25 mg/kg/day based on its effective dosage in SARS-CoV-infected mice²⁰. In this study, hamsters were intra-nasally challenged with 10⁴ PFU SARS-CoV-2 before four consecutive daily dosages beginning at 6 hpi. Expectedly, the DMSO-treated control hamsters developed the clinical signs of lethargy, ruffled fur, hunched back posture and rapid breathing starting from 2 days post infection (d.p.i.), whereas the hamsters treated with either RBC or remdesivir did not develop any clinical sign. At day 4 post-challenge, studies were conducted to examine whether the drug conferred protection against SARS-CoV-2 challenge by reducing the viral loads in the upper respiratory tract (nasal turbinate) and lower respiratory tract (lung). Apparently, Pylorid decreased viral RNA load in both nasal turbinate (p <0.05) and lung tissues (p <0.01) for ˜1 log and 1.5 logs, respectively (FIG. 3A). Consistently, suppression of live SARS-CoV-2 particles in respiratory tract were confirmed in both remdesivir and Pylorid groups (FIG. 3B).

Increased secretion of the pro-inflammatory cytokines and chemokines is associated with the severity of afflicted COVID-19 patients²¹. To ascertain if the therapeutic effect of Pylorid relieved the virus-induced cytokine storm, the expression levels of interleukin 10 (IL-10) and tumour necrosis factor alpha (TNF-α) were determined, prognosis markers of severe COVID-19 cases, as well as representative pro-inflammatory cytokines and chemokines including interferon α (IFN-γ), C-C Motif Chemokine Ligand 22 (CCL22) and C-C chemokine receptor type 4 (CCR4)²². Intriguingly, mRNA expression of IFN-γ (p<0.05), IL-10 (p<0.001) and CCR4 (p<0.05) were remarkably diminished in the hamster group receiving Pylorid treatment, whereas those in the remdesivir group were generally lower but statistically non-significant (except IL-6) when compared with the vehicle (DMSO) group (FIG. 3C).

To provide a clear monitoring of the disease, histological examination of hematoxylin and eosin (H&E)-stained lung tissue was carried out at 4 d.p.i. Significant amelioration of lung damage was observed after RBC treatment (data not shown). In the DMSO control group, a large area of consolidation and massive alveolar space mononuclear cell infiltration and exudation were identified in animal lungs, as well as moderate severity of bronchiolar epithelial cell death. In addition, endothelium and vessel wall mononuclear cells infiltration was observed in the pulmonary blood vessels. Lung tissues in the remdesivir treatment group exhibited improved morphology but a mild degree of bronchiolar wall infiltration and vessel wall infiltration (FIG. 3E). After RBC treatment, however, only slight alveolar wall thickening and mild peribronchiolar infiltration were detected, without visible blood vessel inflammatory changes (data not shown). Immunofluorescence staining indicated diminished N protein expression in alveolar tissue, being mainly expressed in focal bronchiolar epithelial cells of hamster lungs after treatment with remdesivir and RBC (data not shown and FIG. 3F). Collectively, the data thus demonstrate the effectiveness of RBC by disrupting the SARS-CoV-2 replication cycle and virus-associated pneumonia in vivo Pylorid is a potent irreversible inhibitor of SARS-CoV-2 helicase.

Previous studies demonstrated that bismuth(III) drugs function via “shotgun” mechanism, i.e., targeting multiple biological pathways through binding to key proteins, in particular zinc-containing proteins^{23,24}. Given that zinc is frequently incorporated as zinc-fingers or zinc-binding domains into several essential nonstructural proteins of coronavirus, e.g., PLpro cysteine protease²⁵, RNA-dependent RNA polymerase⁹ and helicase²⁶, the hypothesis was that bismuth(III) may functionally inactive these enzymes, thus prohibiting SARS-CoV-2 viral replication. As a proof-of-principle, SARS-CoV-2 helicase was selected as one of the feasible targets to investigate whether bismuth(III) compounds could inactivate the enzyme.

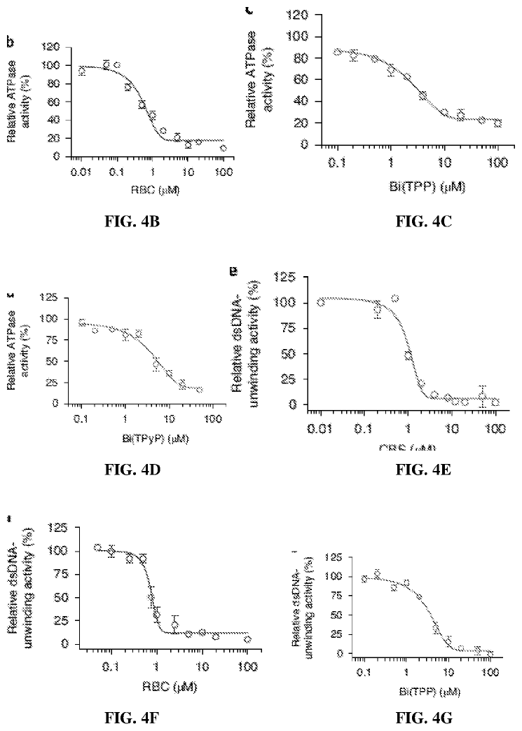

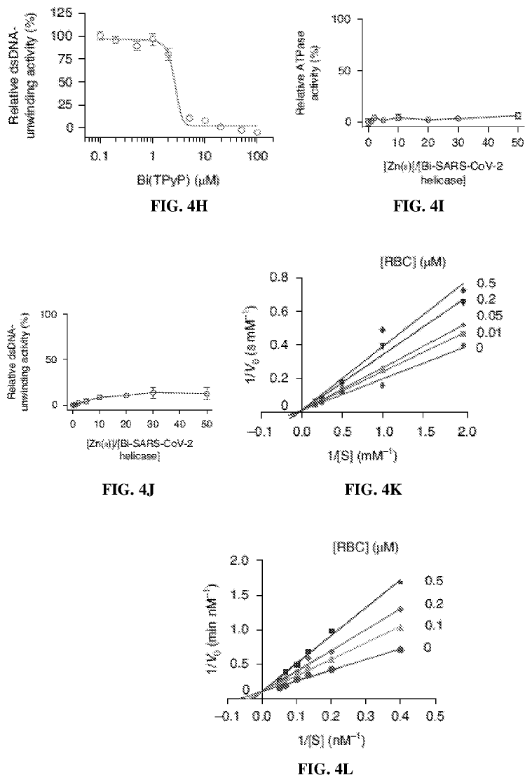

Helicases are motor proteins that serve to convert NTP to NDP and inorganic phosphate (Pi) during ssNA translocation and dsNA separation and unwind both dsRNA and dsDNA with a 5′-ss tail along the polarity of 5′ to 3′^{27,28}. To explore the potential role of Pylorid on SARS-CoV-2 helicase, the full-length protein, which contains a N-terminal zinc-binding domain (ZBD) and a C-terminal helicase domain (HEL) was first overexpressed and purified (FIG. 3D). Studies first examined whether bismuth(III) compounds inhibited the ATPase activity of SARS-CoV-2 helicase by a typical phosphate release assay²⁹, where the phosphate released due to ATP hydrolysis was presented as a relative percentage of the ATPase activity with or without bismuth(III) compounds addition. As shown in FIG. 4A-4D, the ATPase activity was significantly decreased as the concentrations of bismuth(III) increased, with the activity being inhibited, ultimately, over 90%. The half-maximum inhibitory concentration (IC₅₀) values were calculated to be 1.88±0.12, 0.69 0.12, 2.39±0.02 and 4.68±1.39 μM for De-Nol, Pylorid, BiTPP and Bi(TPyP), respectively (Table 2), indicative of the effective inhibition of ATPase activity of SARS-CoV-2 helicase by Pylorid and relevant bismuth(III) compounds.

TABLE 2
Inhibitory potency of the selected compounds on SARS-CoV-2
helicase. Summary of IC₅₀ of the selected compounds towards
ATPase and DNA-unwinding activity of SARS-CoV-2 helicase

| | IC₅₀ (μM) | |
|---|---|---|
| Compound* | ATPase activity | DNA-unwinding activity |
| De-Nol | 1.88±0.12 | 1.24±0.02 |
| Pylorid | 0.69±0.12 | 0.70±0.13 |
| Bi(TPP) | 2.39±0.02 | 3.69±0.26 |
| Bi(TPyP) | 4.68±1.39 | 2.64±0.16 |
| Ridaura | 1.20±0.02 | 0.57±0.03 |
| Au(PEt₃)Cl | 0.23±0.01 | 0.34±0.07 |

*The measure of drug was based on metal content

Additional studies next investigated the effects of the four bismuth compounds on the duplex-unwinding activity of SARS-CoV-2 helicase by an established fluorescence resonance energy transfer (FRET)-based assay¹⁶. The DNA-duplex substrate was prepared by annealing an oligomer with a Cy3 fluorophore at the 3′ end and a BHQ-2 quencher at the 5′end. The proteins and DNA-duplex were equilibrated in the presence of varying concentrations of bismuth compounds before fluorescence titration. In the absence of bismuth(III) compounds, signal intensity of the DNA-duplex increased drastically owing to the unwinding of the Cy3 strand from DNA-duplex via helicase. In contrast, the fluorescence increased much less evidently under increasing concentrations of bismuth(III) compounds, indicative of the inhibition on duplex-unwinding in a dose-dependent manner (FIG. 4E-4H) Similarly, the IC₅₀ values of the compounds against duplex-unwinding activity of the enzyme were measured to be 1.24±0.02 μM for De-Nol, 0.74±0.13 μM for Pylorid, 3.69±0.26 μM for Bi(TPP) and 2.64±0.16 μM for Bi(TPyP) (Table 2). Significantly, such inhibition was irreversible as the supplementation of up to 50 molar equivalents of zinc(II) to bismuth bound SARS-CoV-2 helicase only led to ˜6% ATPase activity and ˜13% duplex-unwinding activity being restored, indicating a limited ability of zinc(II) to compete with bismuth(III) for SARS-CoV-2 helicase (FIG. 4I-4J). From an enzyme kinetic perspective, increasing concentrations of Pylorid barely changed the maximum velocity (Vₘₐₓ) value of around 42.05±2.78 mM/s, whereas an increase of apparent Michaelis-Menten constant (Kₘ) from 5.51 to 12.74 mM, indicative of a competitive inhibition on ATPase activity of SARS-CoV-2 helicase (FIG. 4K-4L). Inhibition constant (Kᵢ) of Pylorid against the helicase ATPase activity was estimated to be 0.97±0.11 μM. Similarly, Pylorid exhibited a competitive mode of inhibition on helicase duplex-unwinding activity with an unchanged Vₘₐₓ value around 20.53±1.56 nM/min, increasing Kₘ values from 42.21 nM to 91.77 nM, and a Kᵢ value estimated as 0.39±0.07 μM. The combined data demonstrate that Pylorid serves as a potent irreversible inhibitor of SARS-CoV-2 helicase.

Pylorid Binds to SARS-CoV-2 Helicase and Releases Zinc Ions from ZBD

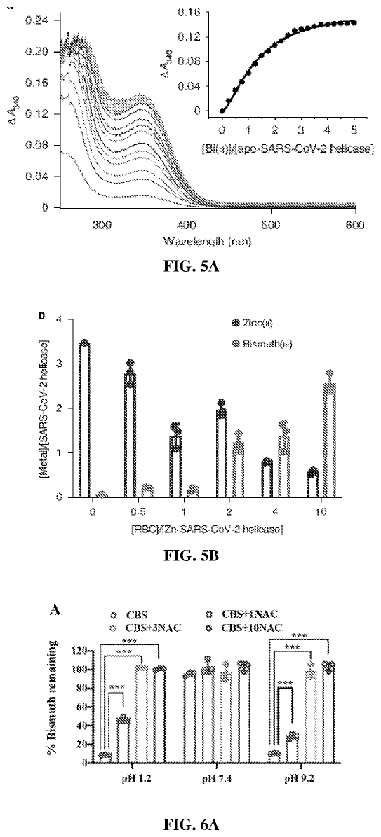

Structural analysis reveals that SARS coronavirus helicase contains three canonical zinc-fingers, including Zinc Finger 1 (Cys5, Cys8, Cys26, Cys29), Zinc Finger 2 (Cys16, Cys19, His33, His39) and Zinc Finger 3 (Cys50, Cys55, Cys72, His75)³⁰. Given the high thiophilicity of bismuth(III), subsequent studies therefore investigated whether bismuth(III) competes with the zinc(II) in zinc-finger sites by UV-vis spectroscopy. To rule out the interference of ranitidine in Pylorid, a colorless bismuth compound, Bi(NTA), was prepared and then titrated to the apo-form of SARS-CoV-2 helicase. Addition of bismuth(III) to apo-SARS-CoV-2 helicase led to the appearance and increase of an absorption band at ˜340 nm, a characteristic of Bi—S ligand-to-metal charge transfer (LMCT) band. As shown in FIG. 5A, the absorption intensities at 340 nm increased, and then plateaued at a molar ratio of [Bi(III)]/[SARS-CoV-2 helicase] of 3, with a dissociation constant (K_{d}) of 1.38±0.05 μM as determined by fitting data with Ryan-Weber nonlinear equation. The results suggest that three bismuth(III) ions bind per SARS-CoV-2 helicase and the cysteine residues in zinc finger sites are involved in the binding.

Next, the question was asked whether binding of bismuth(III) to SARS-CoV-2 helicase resulted in zinc(II) release by inductively-coupled plasma mass spectrometry (ICP-MS). Utilizing equilibrium dialysis, the studies showed that ˜3.46 molar equivalents of Zn(II) bound to SARS-CoV-2 helicase. The titration of Pylorid to SARS-CoV-2 helicase resulted in a decrease in the stoichiometry of Zn(II) ions, accompanied by an increase in that of bismuth(III) to SARS-CoV-2 helicase, eventually, ˜2.90 molar equivalents of Zn(II) were displaced whereas ˜2.73 molar equivalents of bismuth(III) bound to the enzyme (FIG. 5B). The data confirmed that the inhibition of SARS-CoV-2 helicase by Pylorid was attributable to the displacement of Zn(II) in SARS-CoV-2 helicase by Bi(III) ions.

Discussion

Metal compounds have historically been used as antimicrobial agents; however, their utility for antiviral therapy has rarely been explored. The present studies identify Pylorid, a well-tolerated and efficacious anti-H. pylori infection and anti-ulcer drug³¹, has been identified as a potent anti-SARS-CoV-2 activity both in vitro and in vivo. Its potency in the established hamster model for COVID-19 is comparable to or even better than that of remdesivir, which has been approved by the US for emergency use for COVID-19 treatment despite its long-term side effects remain undetermined. The well-characterized safety profile of Pylorid may facilitate its immediate use in clinical trials of COVID-19 patients. The gastrointestinal tract is generally believed to be a potential transmission route and target organ of SARS-CoV-2³², while Pylorid maintains its known good pharmacological activity with the digestive tract's environment. The examination of Pylorid on colinic (Caco2) cells demonstrated its potent activity to suppress SARS-CoV-2 replication (FIG. 1A-1P), which may support the use of Pylorid to restrict virus-induced gastrointestinal manifestations and potential fecal-oral transmission of COVID-19.

Increasing evidence including a recent finding in a randomized trial suggest the advantage of combination therapy targeting multiple steps in the virus life cycle of SARS-CoV-2. Triple therapy consisting of Beteferon, lopinavir/ritonavir and ribavirin achieved significantly faster viral clearance and clinical improvement than monotherapy with lopinavir/ritonavir³³. The multi-system manifestations of COVID-19 infection are caused by the combination of virus-induced cell damage and immunopathologies with dysregulated inflammatory activity. The dysregulated cytokine storm, going hand in hand with a compromised circulatory system, leads to fulminant multi-organ dysfunction affecting lungs, heart, kidneys, nerves, muscles, gastrointestinal tract and brain³⁴. This study has demonstrated the possibility of multi-target inhibition on SARS-CoV-2 by Pylorid (FIG. 2C). This study shows that both the entry and post-entry steps of the SARS-CoV-2 replication cycle are targeted by RBC (FIGS. 2C and 2D). Helicase was selected as an illustrative example to demonstrate the in vitro interaction of RBC with a viral enzyme, that is, irreversibly disrupting enzyme function by the release of vital zinc(ii) and possibly forming a non-functional metallodrug-bound enzyme in SARS-CoV-2-infected cells (FIGS. 4A-4L and 5A-5B). Vero E6 cells were utilized for EC50 measurements; these do not express TMPRSS2, which is a major entry determinant of SARS-CoV-244. Apparently, higher inhibitory efficiency has been achieved by RBC in Calu-3 cells (TMPRSS2+) than that in Vero E6 cells (TMPRSS2-), indicating that RBC interferes with the TMPRSS2-primed virus entry (P<0.01, FIG. 2F) Given that key motifs such as zinc fingers in viral enzymes are highly conserved, Pylorid may serve as a broad-spectrum inhibitor against coronavirus¹⁶. The high selectivity index and approved safety of Pylorid highlight the potential of this drug to be rapidly adopted for the treatment of COVID-19 disease after further clinical validation.

Oral Administration of Bismuth Drugs in Combination with Thiol-Containing Drugs for Broad-Spectrum Anti-Coronavirus Therapy

RBC and other related bismuth drug(s), e.g., colloidal bismuth subcitrate (CBS) and bismuth salicylate (BSS), are orally administered anti-ulcer drugs that precipitates in gastric juice (pH 1-3) to form a protective coating on the ulcer craters and prevents the erosion by gastric secretion⁽⁸⁾. This may lead to reduced systemic absorption and concentration in the lungs which is the primary site of coronavirus (CoV) infections. Additional studies were conducted to stabilize bismuth drugs acidic conditions so that the uptake of bismuth and their anti-SAR-CoV-2 efficacy could be maintained or enhanced. Studies sought to evaluate whether thiol-containing small molecules may prevent hydrolyzation of bismuth drugs under acidic conditions and improve the systemic absorption of bismuth drugs and consequently, efficacy.

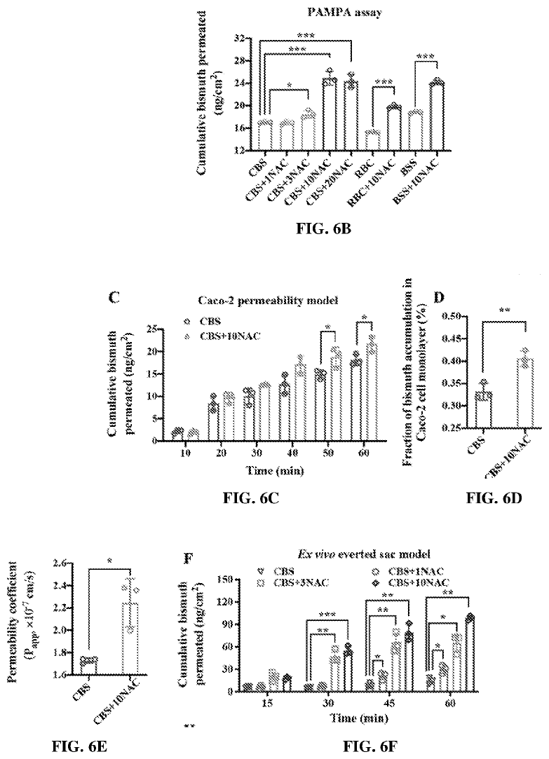

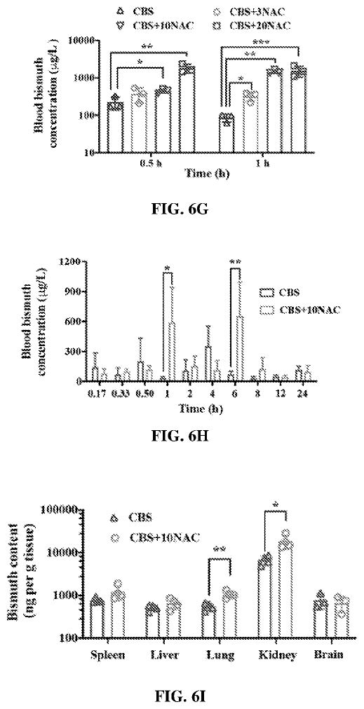

Three thiol-containing drugs (N-acetyl cysteine (NAC)), CPL and PCM), were selected to demonstrate the effect of thiol-containing compounds on the efficacy of bismuth drugs. Initial studies validated whether NAC could stabilize bismuth(III) drug, i.e., CBS, in simulated gastric fluid (pH 1.2), Dulbecco's phosphate buffer saline (PBS, pH 7.4) and sodium bicarbonate buffer (pH 9.2). The combinatorial use of CBS with different molar equivalents of (mol eq.) NAC was denoted as CBS+nNAC hereafter. As shown in FIG. 6A, CBS precipitated immediately at pH 1.2 with less than 10% bismuth found in the supernatant after 1 hour. In contrast, NAC prevented the precipitation of CBS in a dose-dependent fashion, with approximately 100% bismuth remaining in the supernatant in the presence of either 3 or 10 mol eq. NAC. In addition, NAC could similarly prevent hydrolysis of CBS even at pH 9.2. Moreover, NAC stabilized other bismuth drugs, including RBC, bismuth salicylate (BSS), and bismuth subgallate (BSG), at low pH, and a series of other thiol-containing drugs, including glutathione (GSH), penicillamine (PCM), captopril (CPL), and thiosalicylic acid (TSA), and could also prevent hydrolysis of CBS under acidic conditions (data not shown).

The bismuth permeability was estimated through the simulated gastrointestinal barrier in the presence of egg lecithin in dodecane (1% w/v) using a modified parallel artificial membrane permeability assay (PAMPA). After reaching equilibrium state in PBS (iso-pH 1.2), the cumulative permeated bismuth was increased from 16.95, 15.24 and 18.80 ng/cm2 to 24.91, 19.77 and 24.13 ng/cm2 for CBS, RBC and BSS, respectively, in the presence of 10 mol eq. NAC (FIG. 6B). This suggests the chemical stability and permeability of CBS as well as related bismuth drugs could be potentially modulated through combined use of a thiol-containing drug.

Further studies characterized bismuth uptake via gastrointestinal segments in the absence or presence of NAC by the human intestinal epithelial cancer cell line (Caco-2)⁽¹⁷⁾ and a modified ex vivo everted gut sac model (at physiological pH 7.4)⁽¹⁸⁾. From Caco-2 permeability assay (FIG. 6C), bismuth intestinal permeation was moderately elevated within 60 min in the presence of 10 mol eq. NAC and the cellular accumulation of bismuth was increased from 0.33% to 0.40% (FIG. 6D). The human intestinal epithelial cell permeability (Papp) of CBS was increased significantly from 1.66×10-7 cm/s to 2.17×10-7 cm/s in the presence of 10 mol eq. NAC (FIG. 6E). The improved intestinal absorption of bismuth by NAC was further demonstrated in the everted sac model that the cumulative bismuth permeated was remarkably boosted from 14.68 to 30.21, 64.98 and 98.61 ng/cm2 within 60 min when CBS was used in combination with 1, 3 and 10 mol eq. NAC, respectively (FIG. 6F). Collectively, the data demonstrates that the oral absorption of bismuth drug could be potentially improved by co-administering with NAC.

To investigate the potential of a bismuth drug as an oral antiviral agent, pharmacokinetic properties of CBS were evaluated in the absence and presence of NAC. CBS was administered without or with different amounts of NAC to Balb/c mice and found that the blood bismuth concentration was prominently increased from 225.75 to 372.04 and 447.29 μg/L after 0.5-hour exposure, and from 87.27 to 332.76 and 1459.58 μg/L after 1-hour exposure when 150 mg/kg CBS was orally co-administered with 3 mol eq. (180 mg/kg) and 10 mol eq. (610 mg/kg) NAC, respectively (FIG. 6G). The mean blood bismuth concentration was profiled versus time curves after a single oral dose of either CBS (150 mg/kg) and its combination with 10 mol eq. NAC (610 mg/kg) in rats. As shown in FIG. 6H, both CBS and CBS+10NAC group displayed a double-peak profile,⁾. For CBS group, the blood bismuth level decreased from the first peak value of 277.69 μg/L at around 0.5 h, as seen in a previous human study⁽²⁰⁾, and reached to Cmax of 447.06 μg/L at 4 h, with a value of area under the curve over 0 to 12 h (AUC0→12 h) of 1316 h·μg/L. Remarkably, NAC served to increase the peak blood concentration of bismuth to 655.78 μg/L and appeared with a prolonged Tmax (Table 4), resulting in a significant elevation in AUC0→12 h of 2750 h√μg/L.

TABLE 4
Pharmacokinetic parameters of CBS and CBS + 10NAC
after oral administrations (n = 5).

| Pharmacokinetics parameters | CBS*^{#} | CBS + 10NAC* |
|---|---|---|
| Tₘₐₓ (±SD) (h) | 2.53(±2.01) | 4.00(±2.74) |
| Cₘₐₓ (±SD) (μg/L) | 447.06(±132.39) | 758.81(±251.74) |
| AUC_{0→12 h} (±SD) (h · μg/L) | 1316.94 (±474.00) | 2750.00 (±1151.99) |
| AUC_{0→24 h} (±SD) (h · μg/L) | 2324.20 (±759.76) | 3616.20 (±1553.57) |

*Drug dosage used in this study: CBS (150 mg/kg), NAC (610 mg/kg)
^{#}The measurement of bismuth content was based on metal content.

Additionally, NAC significantly improved bismuth accumulation in lung (CBS: 552.15 ng per tissue vs CBS+10NAC: 1056.62 ng per tissue) and kidney (CBS: 6839.76 ng per tissue vs CBS+10NAC: 18788.60 ng per tissue), moderately facilitate the bismuth uptake in other organs i.e., spleen and liver, and had negligible effect on the bismuth uptake in brain, as revealed by the biodistribution profile of bismuth in different organs at 24 hours (FIG. 6I). Taken together, both the in vitro data and the in vivo pharmacokinetics data consistently demonstrated that the co-administration of CBS with NAC led to a remarkably improved bismuth uptake profile in both blood and different organs, which significantly improved the oral availability of bismuth drug for combating SARS-CoV-2 infection.

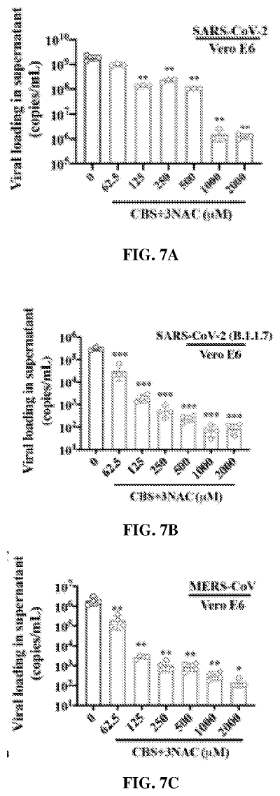

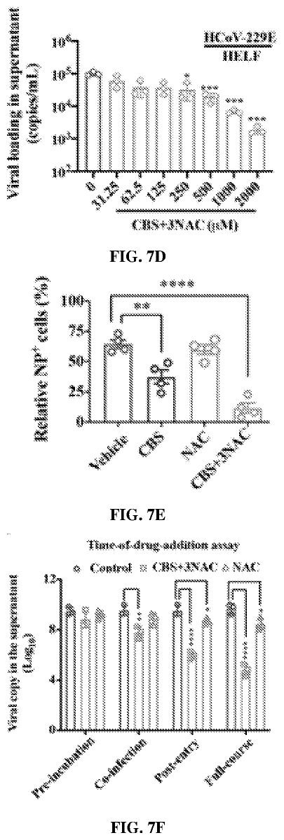

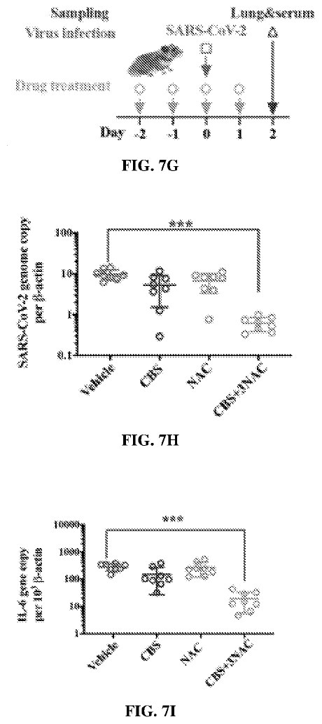

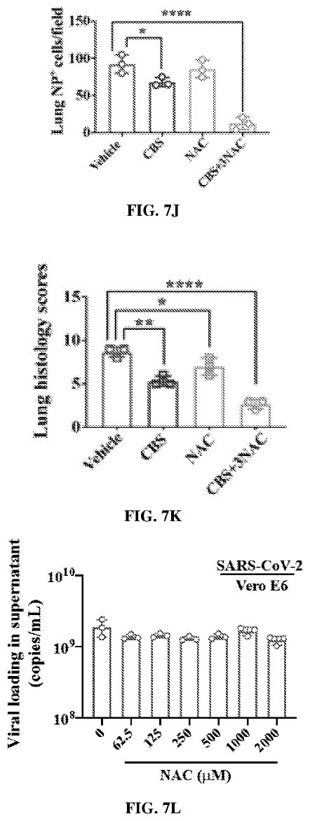

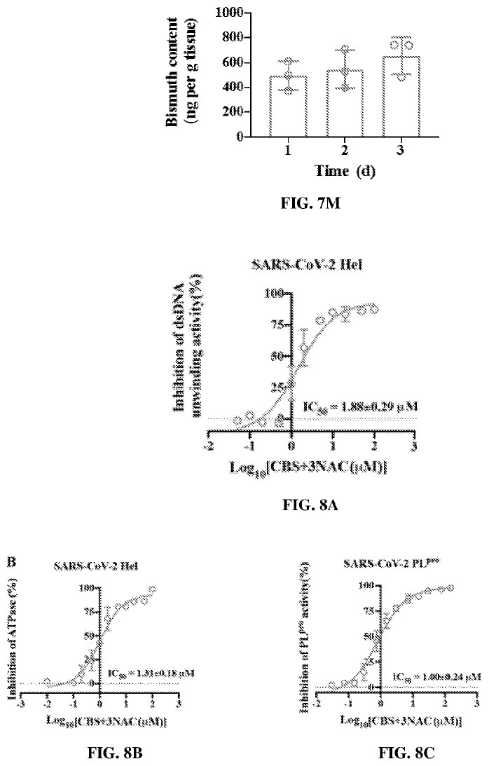

To avoid the potential impact of superabundant NAC on antiviral evaluation⁽²²⁾, Bismuth drugs (CBS and BSS) were co-administered with 3 mol eq. of thiol containing drugs (NAC, CPL and PCM), for the following cell-based and animal-based studies. CBS+3NAC treatment reduced SARS-CoV-2 yield up to >3-log 10 in the Vero E6 cell culture supernatant (FIG. 7A) while NAC alone exhibited negligible anti-SARS-CoV-2 activity at even up to 2000 μM under identical condition (FIG. 7L). The EC₅₀ of CBS+3NAC was estimated to be 5.8 μM according to plaque reduction assay, which was comparable to that of CBS (EC₅₀=4.6 μM)⁽¹⁾, showing the combined use of CBS with NAC did not compromise the antiviral potency of CBS. Significantly, CBS+3NAC treatment remarkably reduced viral yield by about 2 log 10 and about 4 log 10 against SARS-CoV-2 (B.1.1.7)^{(4, 23)} and MERS-CoV-infected Vero E6 cell culture supernatants, respectively, and up to >1.5 log 10 in the cell culture supernatants of hCoV-229E-infected human embryonic lung fibroblasts (HELF). The result indicated that CBS+3NAC may provide a broad-spectrum antiviral option against epidemic and seasonal coronaviruses.

The Enzyme inhibitory activity of CBS+3NAC against SARS-CoV-2 is shown in Table 5.

TABLE 5
Enzyme inhibitory activity of CBS + 3NAC against SARS-CoV-2

| | Inhibitory activity/IC₅₀ (±SD) (uM) * | | | |
|---|---|---|---|---|
| | | | Hel dsDNA- | |
| Compound | PL^{pro} | M^{pro} | unwinding | Hel ATPase |
| CBS + 3NAC | 1.00(±0.24) | 20.10(±1.49) | 1.88(±0.29) | 1.31(±0.18) |
| CBS | 1.02(±0.25) | 22.25(±2.23) | 1.24(±0.02) | 1.88(±0.12) |
| NAC | >150 uM | >450 uM | >150 uM | >150 uM |

* The measurement of bismuth content was based on metal content.
^{#}N.D.: Not determined

Bismuth drugs (CBS and CBS) in combination with thiol containing compounds (NAC, CPL or PCM) suppressed SARS-CoV-2 in Vero E6 cells in a dose-dependent manner (FIG. 11). Viral load in the cell culture supernatant was quantified by qPCR with reverse transcription (RT-qPCR).

Using immunofluorescence staining, the superior antiviral effect of CBS+3NAC was further depicted by prominently lowered viral NP antigen in CBS+3NAC-treated group (11.75%) in comparison to that in non-treated group (64.5%), CBS-treated (37.25%) or NAC-treated group (59.75%) (data not shown and FIG. 7E). The mode of action of CBS+3NAC was explored by a time-of-drug-addition assay in a single viral replication cycle. Treatment with CBS+3NAC robustly hindered the SARS-CoV-2 infection as manifested by 3.54-log 10 and 1.73-log 10 decline in viral load when CBS+3NAC was added during co-incubation and post-entry stages, respectively; while it was observed that CBS+3NAC barely interfered with viral attachment (i.e., pre-incubation, FIG. 7F). Considering the marginal influence of NAC alone on viral replication, it was suggested that CBS+3NAC interferes with multiple steps including SARS-CoV-2 internalization and/or post entry events.

Given the good oral pharmacokinetic profile and prospective antiviral potency of the combination of CBS and NAC in vitro, subsequent studies assessed it's in vivo efficacy in a well-established golden Syrian hamster model⁽²⁴⁾. A pilot study showed that the bismuth content in lung could be well accumulated when CBS+3NAC was administered to mice for 3 consecutive days (FIG. 7M). To achieve optimal antiviral performance, multiple doses of bismuth drugs were given in the infection model. Groups of hamsters were orally administered with aliquot of aqueous solution of CBS+3NAC, CBS, NAC, and water (as a vehicle control), respectively, on day −2, −1, and 6 hours before intranasal challenge of SARS-CoV-2 on day 0, and day 1 post infection (FIG. 7G). The viral loads in the lung in respective group were then determined 2-day post infection (dpi) when the viral loads escalated with prominent histopathological changes. There appeared a 15.87-fold reduction in pulmonary SARS-CoV-2 RNA copies in CBS+3NAC group compared with vehicle group, with significant difference (P<0.0001, Kruskal-Wallis with Dunn's multiple comparison test) between them (FIG. 7H). No statistically significant difference was observed among vehicle, CBS and NAC group. Additionally, lung IL-6 gene expression was determined to mirror the potential respiratory failure and adverse clinical outcome after virus infection. As shown in FIG. 7I, CBS+3NAC treatment led to a significant decreased IL-6 level by 14.4 folds compared with that from the vehicle group, whereas CBS-treatment also caused lowered but statistically non-significant change in IL-6 level. Immunofluorescence staining assay concreated the in vivo anti-SARS-CoV-2 potency of CBS+3NAC as evidenced by a 7.49-fold reduction in SARS-CoV-2-NP expression in alveolar tissue of hamster lungs after treatment with CBS+3NAC (data not shown and FIG. 7J).

Consistently, at the end of the experimental period, signs of lethargy, ruffled fur, hunched back posture and rapid breathing occurred in infected hamsters in vehicle group, whereas these adverse clinical signs and symptoms were significantly ameliorated in CBS+3NAC-treated group and mildly mitigated in CBS-treated group. The severity of lung damage was further examined by performing histological examination of hematoxylin and eosin (H&E) staining in hamster lung tissue. Infected hamsters receiving vehicle developed large areas of consolidation, cell infiltrations in endothelium of blood vessel as well as peribronchiolar regions (data not shown). In contrast, these severe pathological changes were greatly prevented in CBS+3NAC-treated hamsters (data not shown) as revealed by the estimated lung histology scores diminished from 8.67 to 5.33 and 2.66, respectively (FIG. 7K), suggesting oral treatment of CBS+3NAC mitigated the risk of progression to severe disease and accelerated recovery. NAC partially relieved lung pathology too, probably due to its capacity to loosen thick mucus with chronic bronchopulmonary disorders^{(15,25)}. Importantly, NAC may broaden the therapeutic time window of CBS+3NAC in either viral phase or inflammatory phase due to its antioxidant activity⁽¹⁵⁾.

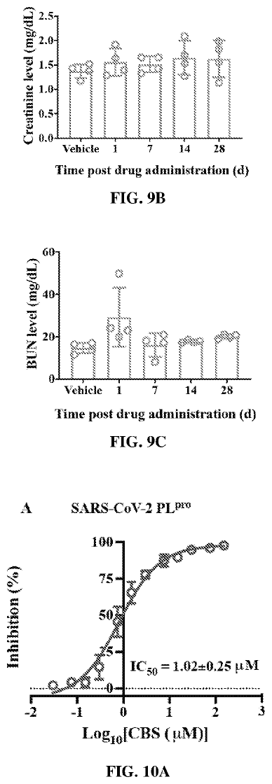

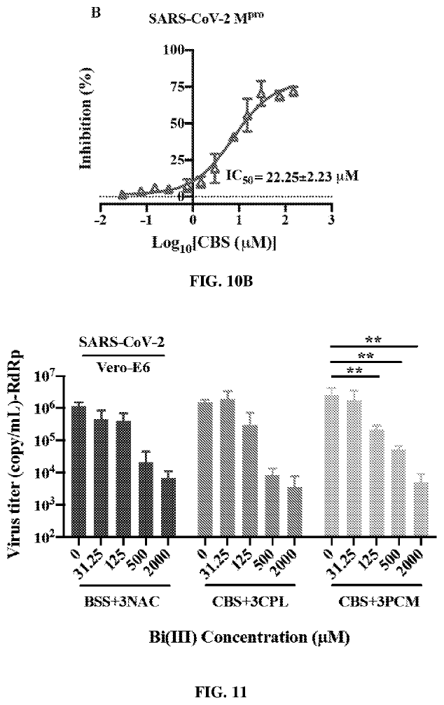

In addition, CBS+3NAC was administered at converted dosage on the basis of body surface area to uninfected Balb/c mice under identical therapeutic condition and found only slightly elevated but reversible change in the level of blood urea nitrogen (BUN) and creatinine while no other pathogenic signs were observed (FIG. 9A-9C). Collectively, the studies demonstrate that, being co-administered with thiol-containing drug, i.e., NAC, bismuth drug i.e., CBS, could be promisingly transformed into an orally available antiviral agent and therefore, robustly reduces viral RNA and pathogenesis of SARS-CoV-2 in vivo.

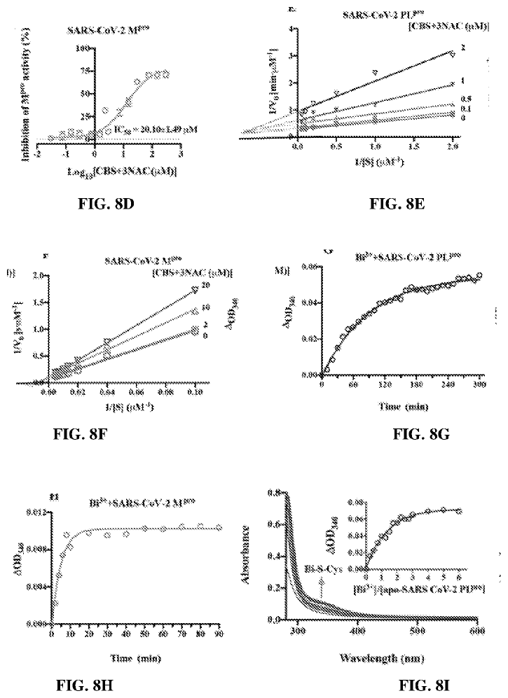

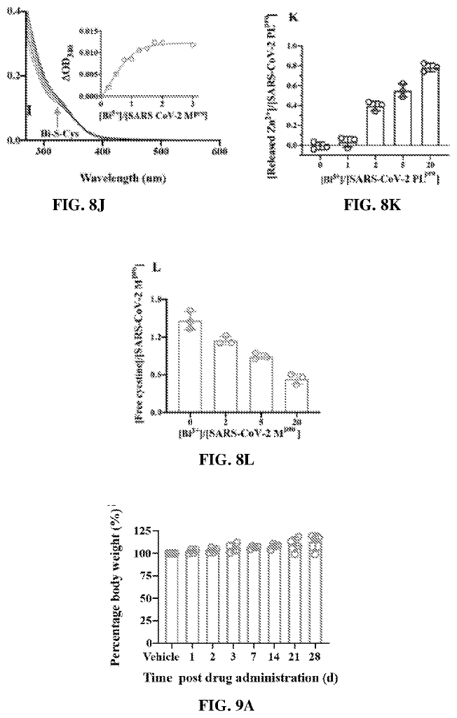

Previous studies showed that bismuth drugs could feasibly target Zn2+-cysteine complexes of proteins in microbes such as the structural zinc-finger domain of SARS-CoV-2/SARS-CoV helicase (Hel, Nsp13)^{(1, 26)}, catalytic zinc active site of NDM-1⁽²⁷⁾ and zinc-binding chaperonin GroES^{(28, 29)} and cysteine protease such as caspases 3 and caspase 9⁽³⁰⁾. The comparable inhibitory effects of CBS+3NAC to CBS on SARS-CoV-2 Hel duplex unwinding activity were verified with IC50 of 1.24 μM for CBS, 1.88 μM for CBS+3NAC and ATPase activity with IC501.88 μM for CBS, and 2.3 μM for CBS+3NAC (FIGS. 8A and 8B). Subsequent studies investigated the potential inhibition of CBS+3NAC on the two distinctive conserved cysteine proteases encoded by the SARS-CoV-2 genome, papain-like protease (PL^{pro}, a domain within Nsp3) possessing a conserved structural Zn2+ in the finger subdomain and chymotrypsin-like main protease (Mpro, Nsp5), both of which are requisitely responsible for the proteolytic cleavage of the two large replicase polyproteins (ORF1a and 1ab) for the viral genome replication⁽³¹⁾, and some biological activities beyond. By using fluorescence resonance energy transfer (FRET)-based cleavage assays, the activity of SARS-CoV-2 PL^{pro} and SARS-CoV-2 M^{pro} were assessed with a peptide substrate of RLRGG↓-AMC and Dabcyl-KTSAVLQ↓SGFRKM-E(Edans)-NH2 respectively. As shown in FIG. 8C, 8D and FIG. 10A-B, CBS+3NAC and CBS inhibited SARS-CoV-2 PL^{pro} with IC50 of 1.00 μM and 1.02 μM respectively and SARS-CoV-2 M^{pro} with IC50 of 21.10 μM, and 22.25 μM respectively in a dose-dependent fashion while NAC exhibited negligible inhibitory at comparable concentrations (Table 6), suggesting the inhibitory effect stemmed from bismuth ion. Increasing concentrations of CBS+3NAC revealed an unchanged value of maximum velocity (Vmax) value at 12.48±0.53 nM/s, whereas an increase in apparent Michaelis—Menten constant (Km) from 176.4 to 320.0 nM was observed, indicative of a typical competitive inhibition of CBS+3NAC on SARS-CoV-2 Mpro activity with an inhibition constant (Ki) of 6.20±0.40 μM; while CBS+3NAC showed a mixed inhibition on SARS-CoV-2 PL^{pro}, possibly owing to the binding of Bi3+to its allosteric site(s) as well as to its active site cysteine (FIGS. 8E and 8F).

TABLE 6
Cytotoxicity and antiviral activity of CBS + 3NAC

| | CBS + 3NAC* |
|---|---|
| CoV/cell line | EC₅₀(±SD) (uM)) |
| SARS-CoV-2/Vero E6 | 5.8(±0.5) |
| SARS-CoV-2 (B.1.1.7)/Vero E6 | 7.4(±1.2) |
| MERS-CoV/Vero E6 | 11.2(±2.3) |
| HCoY-229E/HELF | 21.4(±5.6) |

″The measurement of bismuth content was based on metal content.

The binding of Bi³⁺ to the cysteine residues of SARS-CoV-2 PL^{pro} and SARS-CoV-2 M^{pro} were monitored as evidenced by appearance of characteristic Bi—S ligand-to-metal charge transfer (LMCT) band at ˜340 nm when 20 mol eq. Bi³⁺ was titrated to respective protein (FIGS. 8G and 8H) with t1/2(PL^{pro}) of 62.07 min, and t1/2(M^{pro}) of 3.38 min. Upon the escalation of Bi3+, the absorption at 340 nm increased and then levelled off at a molar ratio of [Bi3+]/[SARS-CoV-2 PL^{pro}] of ˜3 and [Bi3+]/[SARS-CoV-2 Mpro] of ˜1, with estimated dissociation constant (Kd) of 1.13 μM and 0.60 μM, respectively (FIGS. 3I 8I and 3J 8J). The binding of Bi3+led to the release of ˜0.78 eq. Zn²⁺ from SARS-CoV-2 PL^{pro}, which in part contributed to the inhibition of its activity. Additionally, the amount of free cysteine in SARS-CoV-2 M^{pro} was found to decrease by ˜1 eq. upon the binding of Bi3+as determined by Ellman's assay. Coincided with previous data (FIG. 7F), these results imply that bismuth drugs, i.e., CBS+3NAC, block multiple biological events during the post-entry stage, through binding and functionally inactivation of crucial cysteine protease, i.e., PL^{pro}, Mpro and Hel, eventually leading to the prohibition of coronavirus replication.

Together, these studies demonstrate that a combination therapy which includes a metallodrug CBS and a thiol-containing drug such as NAC can serve as an orally administrated broad-spectrum anti-CoV regimen through targeting multiply crucial viral enzymes.

### REFERENCES

1 Zhou, et al. Nature 579, 270-273, doi:10.1038/s41586-020-2012-7 (2020).
2 Stadler, et al. SARS—Nat Rev Microbiol 1, 209-218 (2003).
3 Wrapp, et al. Science 367, 1260-1263, doi:10.1126/science.abb2507 (2020).
4 Lan, et al. Nature, doi:10.1038/s41586-020-2180-5 (2020).
5 Shang, et al. S Nature, doi:10.1038/s41586-020-2179-y (2020).
6 Sheahan, et al. Sci Transl Med, doi:10.1126/scitranslmed.abb5883 (2020).
7 Zhang, et al. Science, doi:10.1126/science.abb3405 (2020).
8 Jin, et al. Nature, doi:10.1038/s41586-020-2223-y (2020).
9 Gao, et al. Science, doi:10.1126/science.abb7498 (2020).
10 Sanders, et al JAMA, doi:10.1001/jama.2020.6019 (2020).
11 Serafin, et al. Int J Antimicrob Agents, 105969, doi:10.1016/j.ijantimicag.2020.105969 (2020).
12 Munster, et al. bioRxiv, 2020.2003.2021.001628, doi:10.1101/2020.03.21.001628 (2020).
13 https://www.nih.gov/news-events/news-releases/nih-clinical-trial-shows-remdesivir-accelerates-recovery-advanced-covid-19
14 Wang, et al. Lancet 395, 1569-1578, doi:10.1016/S0140-6736(20)31022-9 (2020).
15 Chan, et al. Lancet 395, 514-523, doi:10.1016/S0140-6736(20)30154-9 (2020).
16 Yang, et al. Angew. Chem. Int. Ed. Engl. 46, 6464-6468, doi:10.1002/anie.200701021 (2007).
17 Pipkin, et al. Pharmacoepidemiol. Drug Saf. 5, 399-407, doi:10.1002/(sici)1099-1557(199611)5:6<399::Aid-pds243>3.0.Co;2-4 (1996).
18 KOCH, et al. Br. J. Clin. Pharmacol. 42, 201-205, doi:10.1046/j.1365-2125.1996.03929.x (1996).
19 Chan, et al. Clin. Infect. Dis., doi:10.1093/cid/ciaa325 (2020).
20 Wang, et al. Cell Res 30, 269-271, doi:10.1038/s41422-020-0282-0 (2020).
21 Tay, et al Nat. Rev. Immunol., doi:10.1038/s41577-020-0311-8 (2020).
22 Huang, et al. Lancet 395, 497-506, doi:10.1016/S0140-6736(20)30183-5 (2020).
23 Wang, et al. Nat Commun 9, 439, doi: 10.1038/s41467-018-02828-6 (2018).
24 Cun, et al P Natl Acad Sci USA 107, 4943-4948, doi:10.1073/pnas.0913970107 (2010).
25 Baez-Santos, et al Antivir Res 115, 21-38, doi:10.1016/j.antivira1.2014.12.015 (2015).
26 Jia, et al. Nucleic Acids Res 47, 6538-6550, doi:10.1093/nar/gkz409 (2019).
27 Patel, et al. J. Biol. Chem. 281, 18265-18268, doi:10.1074/jbc.R600008200 (2006).
28 Keum, et al Biochem Pharmacol 84, 1351-1358, doi:10.1016/j.bcp.2012.08.012 (2012).
29 Tanner, et al. J. Biol. Chem. 278, 39578-39582, doi:10.1074/jbc.C300328200 (2003).
30 Jia, et al. Nucleic Acids Res. 47, 6538-6550, doi:10.1093/nar/gkz409 (2019).
31 Bardhan, et al Gut 41, 181-186, doi:10.1136/gut.41.2.181 (1997).
32 Lin, L. et al. Gastrointestinal symptoms of 95 cases with SARS-CoV-2 infection. Gut 69, 997-1001, doi:10.1136/gutjnl-2020-321013 (2020).
33 Hung, et al. Lancet, doi:10.1016/S0140-6736(20)31042-4 (2020).
34 Roberts, M. et al. Covid-19: a complex multisystem clinical syndrome. at https://blogs.bmj.com/bmj/2020/05/01/covid-19-a-complex-multisystem-clinical-syndrome/ (2020).
35 Li, et al Curr. Opin. Chem. Biol. 16, 74-83, (2012).
36 Wang, et al. B iScience 23, 101054, doi:https://doi.org/10.1016/j.isci.2020.101054 (2020).
37 Chu, et al. Lancet Microbe 1, e14-e23, doi:https://doi.org/10.1016/S2666-5247(20)30004-5 (2020).
38 Yuan, et al. Nat. Commun. 10, 120, doi:10.1038/s41467-018-08015-x (2019).
39 Yuan, et al. J. Antimicrob. Chemother. 71, 2489-2497, doi:10.1093/jac/dkw194 (2016).
40 Chan, et al. T Antiviral Res. 160, 38-47, doi:https://doi.org/10.1016/j.antiviral.2018.10.007 (2018).
41 Yuan, et al. Antimicrob. Agents Chemother. 59, 4082-4093, doi:10.1128/aac.00306-15 (2015).
42 Chan, et al. J. Clin. Microb. 58, e00310-00320, doi:10.1128/jcm.00310-20 (2020).
1. Yuan, et al. Nat. Microb. 5, 1439-1448((2020).
2. World Health Organization. Coronavirus Disease (COVID-19) Situation Reports. https://www.who.int/emergencies/diseases/novel-coronavirus-2019/situation-reports., (2020).
3. Graham, Science 368, 945-946 (2020).
4. Emerging SARS-CoV-2 variants. Centers for Disease Control and Prevention. https://www.cdc.gov/coronavirus/2019-ncov/more/science-and-research/scientific-brief-emerging-variants.html#_ftnl. (2021).
5. Zumla, et al. Nat. Rev. Drug Discov. 15, 327-347 (2016).
6. Beigel, et al. N. Engl. J. Med. 383, 994 (2020).
7. Goldman, et al. N. Engl. J. Med. 383, 1827-1837 (2020).
8. Sun, Curr. Opin. Chem. Biol. 16, 74-83 (2012).
9. D. C. Wolf, C. H. Wolf, D. T. Rubin, Temporal improvement of a COVID-19-positive Crohn's disease patient treated with bismuth subsalicylate. Am. J. Gastroenterol. 115, 1298 (2020).
10. Wang, et al. Acc. Chem. Res. 52, 216-227 (2019).
11. Sadler, et al. Chem. Eur. J. 2, 701-708 (1996).
12. Prescott, et al. Br. Med. J. 2, 1097-1100 (1979).
13. Prescott, et al. Lancet 2, 432-434 (1977).
14. Smilkstein, et al. N. Engl. J. Med. 319, 1557-1562 (1988).
15. Omara, et al. Toxicology 116, 219-226 (1997).
16. Shi, et al. Ther. Clin. Risk Manag. 16, 1047-1055 (2020).
17. Ye, et al. J. Med. Chem. 63, 7695-7720 (2020).
18. Alam, et al. J. Pharm. Pharmacol. 64, 326-336 (2011).
19. Leussink, et al. Arch. Toxicol. 74, 349-355 (2000).
20. Hespe, et al. Lancet 332, 1258 (1988).
21. Riva, et al. Nature 586, 113-119 (2020).
22. Geiler, et al. Biochem. Pharmacol. 79, 413-420 (2010).
23. Kemp, et al. bioRxiv, 2020.2012.2014.422555 (2020).
24. Chan, et al. Clin. Infect. Dis. 71, 2428-2446 (2020).
25. Dekhuijzen, Eur. Respir. J. 23, 629-636 (2004).
26. Yang, et al. Angew. Chem. Int. Ed. 46, 6464-6468 (2007).
27. Wang, et al. Nat. Commun. 9, 439 (2018).
28. S. Cun, H. Sun, A zinc-binding site by negative selection induces metallodrug susceptibility in an essential chaperonin. Proc. Natl. Acad. Sci. U.S.A. 107, 4943 (2010).
29. Cun, et al. J. Biol. Chem. 283, 15142-15151 (2008).
30. Wang, et al. iScience 23, 101054 (2020).
31. Lei, et al. Antivir. Res. 149, 58-74 (2018).

1. A method of treating a subject for a SARS-CoV-2 infection in a subject in need thereof comprising administering the subject a composition comprising a effective amount of one or more Bismuth (III)-containing compounds, an analog thereof, or pharmaceutically acceptable salt thereof in a phramaceutically acceptable carrier, in a therapeutically effective amount to reduce one or more symptoms of a SARS-CoV-2 infection.

2. The method of claim 1, wherein: (a) the effective amount of one or more Bismuth (III)-containing compounds is effective to inhibit the helicase protein of SARS-CoV-2, in the subject (b) wherein the composition is administered in an effective amount to reduce viral replication; and/or (c) wherein the subject is presently suffering from an infection of the SARS-CoV-2.

3. The method of claim 1, wherein the Bismuth (III)-containing compound or pharmaceutically acceptable salt thereof is administered systemically.

4. The method of claim 1, wherein the Bismuth (III)-containing compound or pharmaceutically acceptable salt thereof is administered orally or parenterally.

5. (canceled)

6. The method of claim 1, wherein the Bismuth (III)-containing compound or pharmaceutically acceptable salt thereof is administered in an effective amount to reduce one or more symptoms of a disease, disorder, or illness associated with the coronavirus.

7. The method of claim 6, wherein the symptoms include fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhea, tiredness, nausea, vomiting, and combinations thereof.

8. (canceled)

9. The method of claim 2, wherein the subject has COVID-19.

10. The method of claim 1, wherein the subject has been exposed to the SARS-CoV-2, but is asymptomatic.

11. The method claim 1, wherein the composition comprises one or more compounds selected from the group consisting of: 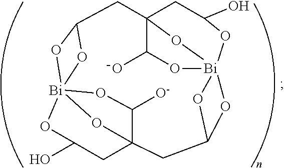
ranitidine bismuth citrate (RBC): 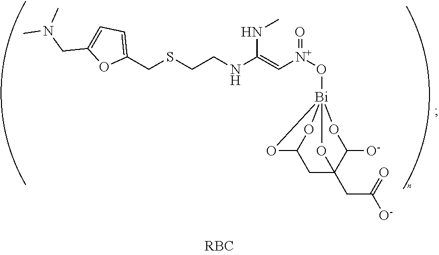
Bi(TPP) (TPP: tetraphenylporphyrinate) 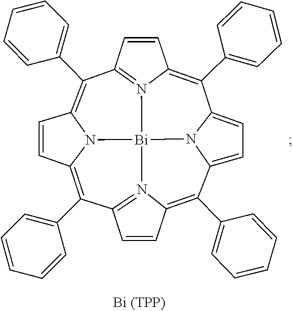
and
Bi(TPyP) (TPyP: tetra(4-pyridyl)porphyrin)

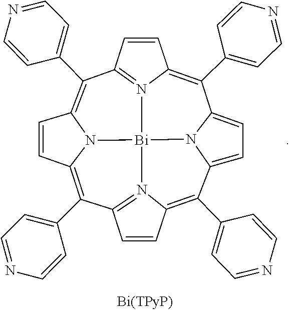

12. The method of claim 1, wherein the composition comprises colloidal bismuth subcitrate (CBS).

13. The method of claim 11, wherein the composition comprises ranitidine bismuth citrate.

14. The method claim 1, wherein the composition is in the form of a tablet for oral administration or in a form suitable for injection.

15. (canceled)

16. A dosage form comprising one or more compounds selected from the group consisting of: 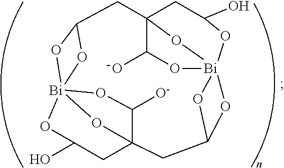
ranitidine bismuth citrate (RBC): 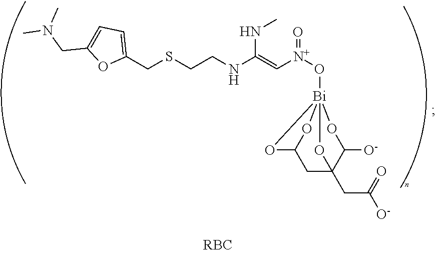
Bi(TPP) (TPP: tetraphenylporphyrinate) 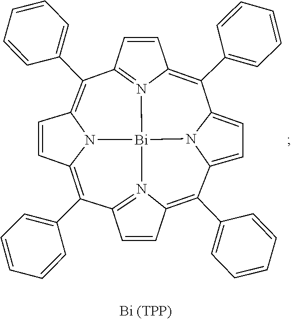
and
Bi(TPyP) (TPyP: tetra(4-pyridyl)porphyrin) 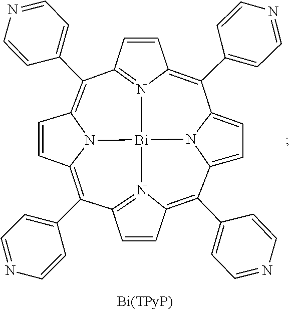
in an effective amount to inhibit Sars-CoV-2 helicase protein, in a subject, following administration, alone or in combination with 3 or 10 mol eq.

17. The dosage form of claim 16, wherein the dosage form is a tablet or capsule.

18. The dosage form of claim 16, wherein the dosage form is an injectable.

19. The dosage form of claim 16, further comprising one or more thiol-containing small molecule compounds.

20. The method of claim 1 further comprising administering one or more thiol-containing small molecule compounds to the subject, or wherein the composition comprises one or more thiol-containing small molecule compounds, wherein the small molecule compound is preferably N-acetyl-cysteine, glutathione, penicillamine (PCM), captopril (CPL), and thiosalicylic acid (TSA).

21. The method of claim 20, wherein the small molecule compound is in an effective amount to stabilize the Bismuth (III) compounds or pharmaceutically acceptable salts thereof at low pH, for example, pH 1.2.

22. The method of claim 21, wherein the one or more thiol-containing small molecule is administered concurrently, or sequentially.

23. The method of claim 20, wherein the thiol containing small molecule is used at a 3 or 10 mol eq. to the bismuth (III) compounds or pharmaceutically acceptable salts thereof.
